(19) United States
(12) Patent Application Publication
(10) Pub. No.: US 2018/0245115 A1
(43) Pub. Date: Aug. 30, 2018

(54) METHOD, KIT AND DEVICE FOR PREPARING GLYCOPROTEIN SUGAR CHAIN

(71) Applicant: SUMITOMO BAKELITE CO., LTD., Tokyo (JP)

(72) Inventor: Masaaki TOYODA, Tokyo (JP)

(73) Assignee: SUMITOMO BAKELITE CO., LTD., Tokyo (JP)

(21) Appl. No.: 15/758,593

(22) PCT Filed: Sep. 27, 2016
(86) PCT No.: PCT/JP2016/078420

§ 371 Date: Mar. 8, 2018

(30) Foreign Application Priority Data
Oct. 9, 2015 (JP) 2015-201064
Oct. 20, 2015 (JP) 2015-206262
Jan. 21, 2016 (JP) 2016-009908
Mar. 18, 2016 (JP) 2016-055369
Apr. 18, 2016 (JP) 2016-082675

(51) Int. Cl. C12P 19/04; C12M 1/40

(52) U.S. Cl. CPC C12P 19/04; C12M 21/18

(57) ABSTRACT

Provided is a method for preparing a glycoprotein sugar chain including: an isolation step of acting a sugar chain-isolating enzyme on a sample which contains a glycoprotein fixed to a solid phase in a container to obtain an isolated product which contains a sugar chain; and a labeling step of adding a labeling reagent to the isolated product in the container to obtain a labeled product which contains a labeled substance of the sugar chain.

### TECHNICAL FIELD

The present invention relates to a method, a kit, and a device for preparing a glycoprotein sugar chain. More specifically, the present invention relates to a method of rapidly preparing a sugar chain from a glycoprotein.

Priority is claimed on Japanese Patent Application No. 2015-201064, filed on Oct. 9, 2015, Japanese Patent Application No. 2015-206262, filed on Oct. 20, 2015, Japanese Patent Application No. 2016-009908, filed on Jan. 21, 2016, Japanese Patent Application No. 2016-055369, filed on Mar. 18, 2016, and Japanese Patent Application No. 2016-082675, filed on Apr. 18, 2016, the contents of which are incorporated herein by reference.

### BACKGROUND ART

A method of recovering a sugar chain isolated from a glycoprotein by allowing a carrier specifically bonded to a sugar chain to capture the sugar chain in order to prepare an isolated sugar chain from a glycoprotein has been known. For example, PTL 1 discloses a method of analyzing a glycoprotein sugar chain, including a step of obtaining a solid phase (specifically, an electrophoretic gel used for electrophoresis or a blotting membrane) by which a glycoprotein is held; a step of treating the solid phase by sugar chain isolating means; a step of obtaining a solution containing a sugar chain by eluting the isolated sugar chain from the solid phase; a step of bringing the solution containing the sugar chain into contact with a capturing carrier specifically bonded to a sugar chain to capture the sugar chain on the capturing carrier; a step of removing materials other than the sugar chain which have not been bonded to the capturing carrier; a step of re-isolating the sugar chain bonded to the capturing carrier to obtain a purified sugar chain sample; and a step of analyzing the sugar chain. According to this method, the re-isolation of the sugar chain is performed by an exchange reaction with a labeling reagent.

### CITATION LIST

### Patent Literature

[PTL 1] Japanese Unexamined Patent Application, First Publication No. 2009-156587

### SUMMARY OF INVENTION

### Technical Problem

In a case where electrophoresis is used as described in PTL 1, it is necessary to carry out a step of visualizing a band of a glycoprotein by performing isotopic labeling or dyeing on the band on a gel or visualizing the band by performing an antigen-antibody reaction or dyeing on the band after being transferred onto a membrane in order to detect a glycoprotein separated by electrophoresis. As described above, it takes a long time to separate a glycoprotein. Further, since components used for visualization are mixed into the glycoprotein, an enzymatic reaction for isolating a sugar chain is affected by the components or the components are mixed into a sugar chain sample after the isolation. Therefore, since such components are required to be separated in order to obtain a sugar chain, it takes more time for the separation. Further, in order to obtain a sugar chain as a modified substance to which a labeling group suitable for the analysis is attached, a labeling step is performed after the sugar chain is temporarily separated from the protein. In this manner, it takes an extremely long time to obtain a sugar chain from a glycoprotein as a modified substance.

Further, a solid phase (for example, protein A sepharose specifically capturing an antibody) capable of fixing a glycoprotein by allowing the glycoprotein to be bonded thereto has been known, but such a solid phase is used exclusively for purification of glycoproteins. In other words, such a solid phase is used for capturing a glycoprotein to be separated from impurities and isolating the captured glycoprotein from the solid phase again. Since the step of isolating a sugar chain from a glycoprotein is performed on the glycoprotein purified in the above-described manner, it also takes a long time to obtain a sugar chain.

In addition, there has been a constant demand for accelerating the preparation of a sugar chain from a glycoprotein. Accordingly, an object of the present invention is to provide a technique for rapidly preparing a labeled sugar chain from a glycoprotein.

### Solution to Problem

The present invention includes the following aspects.

[1] A method for preparing a glycoprotein sugar chain, including: an isolation step of acting a sugar chain-isolating enzyme on a sample which contains a glycoprotein fixed to a solid phase in a container to obtain an isolated product which contains a sugar chain; and a labeling step of adding a labeling reagent to the isolated product in the container to obtain a labeled product which contains a labeled substance of the sugar chain.

[2] The method for preparing a glycoprotein sugar chain according to [1], further including: a pre-treatment step of bringing a pre-treatment agent containing a surfactant into contact with the sample before the isolation step.

[3] The method for preparing a glycoprotein sugar chain according to [1] or [2], in which the isolation step is performed in the presence of a deglycosylation promoter containing an acid-derived anionic surfactant.

[4] The method for preparing a glycoprotein sugar chain according to [3], in which the acid-derived anionic surfactant is selected from the group consisting of a carboxylic acid type anionic surfactant, a sulfonic acid type anionic surfactant, a sulfuric acid ester type anionic surfactant, and a phosphoric acid ester type anionic surfactant.

[5] The method for preparing a glycoprotein sugar chain according to any one of [1] to [4], in which the isolation step is performed in an open system and under heating conditions.

[6] The method for preparing a glycoprotein sugar chain according to any one of [1] to [5], in which the glycoprotein is an antibody, a hormone, an enzyme, or a complex containing these.

[7] The method of preparing a glycoprotein sugar chain according to any one of [1] to [6], in which the solid phase is selected from the group consisting of a cation exchange carrier, a hydrophobic interaction carrier, and an inorganic carrier.

[8] The method for preparing a glycoprotein sugar chain according to any one of [1] to [7], in which the glycoprotein is an antibody, and the solid phase includes a ligand selected from the group consisting of protein A, protein CG protein L, protein H, protein D, and protein Arp, in the surface thereof.

[9] The method of preparing a glycoprotein sugar chain according to any one of [1] to [8], in which the labeling reagent contains 2-aminobenzamide, a reducing agent, and a solvent.

[10] The method of preparing a glycoprotein sugar chain according to [9], in which the reducing agent is picoline borane.

[11] The method of preparing a glycoprotein sugar chain according to [9] or [10], in which the solvent contains a protic compound.

[12] The method of preparing a glycoprotein sugar chain according to [1], in which the solvent further contains an aprotic compound having a boiling point higher than that of the protic compound.

[13] The method of preparing a glycoprotein sugar chain according to any one of [1] to [12], further including: a separation step of performing solid-liquid separation after the isolation step to obtain a separate liquid which contains the isolated product.

[14] The method of preparing a glycoprotein sugar chain according to any one of [1] to [12], further including: a separation step of performing solid-liquid separation after the labeling step to obtain a separate liquid which contains the labeled substance of the sugar chain.

[15] A kit for preparing a glycoprotein sugar chain, including: a solid phase for fixing a glycoprotein; a container for isolating and labeling a sugar chain by holding the solid phase; and a sugar chain-isolating enzyme.

[16] The kit for preparing a glycoprotein sugar chain according to [15], further including: a pre-treatment agent which contains a surfactant; a deglycosylation promoter which contains an acid-derived anionic surfactant; or a labeling reagent.

[17] The kit for preparing a glycoprotein sugar chain according to [16], in which the labeling reagent contains 2-aminobenzamide, a reducing agent, and a solvent.

[18] The kit for preparing a glycoprotein sugar chain according to any one of [15] to [17], in which the solid phase is selected from the group consisting of a cation exchange carrier, a hydrophobic interaction carrier, and an inorganic carrier.

[19] The kit for preparing a glycoprotein sugar chain according to any one of [15] to [18], in which the solid phase includes a ligand selected from the group consisting of protein A, protein G, protein L, protein H, protein D, and protein Arp, in the surface thereof.

[20] A device for preparing a glycoprotein sugar chain including: a container holding portion which holds a container in which a sample that contains a glycoprotein fixed to a solid phase is accommodated; and a reagent introducing unit which introduces a reagent into the container, in which the reagent introducing unit includes a sugar chain-isolating enzyme introducing unit which introduces a sugar chain-isolating enzyme into the container and a labeling reagent introducing unit which introduces a labeling reagent into the container.

[21] The device for preparing a glycoprotein sugar chain according to [20], further including: a solid-liquid separating unit which performs solid-liquid separation on the contents in the container.

[22] The device for preparing a glycoprotein sugar chain according to [20] or [21], further including: a temperature adjusting unit which adjusts the temperature of the contents in the container.

### Advantageous Effects of Invention

According to the present invention, it is possible to provide a technique for rapidly preparing a labeled sugar chain from a glycoprotein.

### BRIEF DESCRIPTION OF DRAWINGS

FIG. 1 is an HPLC spectrum obtained in Comparative Example 1.

FIG. 2 is an HPLC spectrum obtained in Example 1.

FIG. 3 is an HPLC spectrum obtained in Example 2.

FIG. 4 is an HPLC spectrum obtained in Example 3.

FIG. 5 is an HPLC spectrum obtained in Reference Example 1.

FIG. 6 is an HPLC spectrum obtained in Example 4.

FIG. 7 is a graph for comparing the peak area ratios of the HPLC spectra obtained in Comparative Example 1, Reference Example 1, and Example 3.

FIG. 8 is an HPLC spectrum obtained in Example 8.

FIG. 9 is an HPLC spectrum obtained in Example 6.

FIG. 10 is an HPLC spectrum obtained in Example 7.

FIG. 11 is a graph for respectively showing the total value of peak areas of the HPLC spectra obtained in Examples 5 to 7.

FIG. 12 is HPLC spectra obtained in Example 8 (acetic acid concentration of 40% by volume) to Example 11 (acetic acid concentration of 70% by volume).

FIG. 13 is HPLC spectra obtained in Example 7 (acetic acid concentration of 75% by volume) and Example 12 (acetic acid concentration of 80% by volume) to Example 14 (acetic acid concentration of 95% by volume).

FIG. 14 is a graph showing a relationship between the concentration of acetic acid and the total value of peak areas of the HPLC spectra obtained in Examples 7 to 14.

FIG. 15 is an HPLC spectrum obtained in Example 15.

FIG. 16 is a graph showing a relationship between the reaction time and the total value of peak areas of the HPLC spectrum obtained in Example 15.

FIG. 17 is an HPLC spectrum obtained in Comparative Example 2.

FIG. 18 is an HPLC spectrum obtained in Comparative Example 3.

FIG. 19 is an HPLC spectrum obtained in Reference Example 2.

FIG. 20 is an HPLC spectrum obtained in Example 16.

FIG. 21 is an HPLC spectrum obtained in Example 17.

FIG. 22 is a graph showing the total area of peaks in FIG. 21.

FIG. 23 is an HPLC spectrum obtained in Example 18.

FIG. 24 is a view schematically illustrating an example of a device for preparing a glycoprotein sugar chain.

### DESCRIPTION OF EMBODIMENTS

According to a first embodiment, the present invention provides a method for preparing a glycoprotein sugar chain, including: an isolation step of acting a sugar chain-isolating enzyme (sugar chain-decomposing enzyme) on a sample contains a glycoprotein fixed to a solid phase in a container to obtain an isolated product which contains a sugar chain; and a labeling step of adding a labeling reagent (labeling reaction solution) to the isolated product in the container to obtain a labeled product which contains a labeled substance of the sugar chain.

According to the method of the present embodiment, a sugar chain can be extremely rapidly prepared from a glycoprotein in the form (labeled form) of a sample for analysis by isolating a sugar chain on a solid phase without eluting a glycoprotein fixed to the solid phase, and adding a labeling reagent (labeling reaction solution) to the container without separating the isolated product.

Further, according to the method of the present embodiment, the glycoprotein provided in the isolation step is fixed to the solid phase. Examples of the form of fixation in this case include a non-covalent bond (a hydrogen bond or an ionic bond) due to a specific bond; and a covalent bond. In addition, the examples do not include the form in which a glycoprotein is merely held by being applied to an electrophoretic gel or being transferred to a blotting membrane.

[Isolation Step]

In the isolation step, an isolated product is obtained by allowing a sugar chain-isolating enzyme to act on a glycoprotein which is fixed to a solid phase to isolate a sugar chain. It is preferable that the sugar chain-isolating enzyme is allowed to act on a glycoprotein in the presence of a deglycosylation promoter. The present step does not substantially include a protein fragmentation step to be performed by chemical fragmentation or enzymatic fragmentation.

(Sample Containing Glycoprotein Fixed to Solid Phase)

<<Glycoprotein>>

The glycoprotein may be a protein containing at least a sugar chain as a composite component. A glycoprotein sugar chain portion may be of an N-binding type or an O-binding type. Further, a sugar chain portion may have a natural structure or may be artificially modified. In addition, the sugar chain portion may be formed of a neutral sugar chain or an acidic sugar chain. A sugar chain binding site in a glycoprotein may be a site which is the same as a natural product or may be a site to which a sugar chain is not bonded in a natural product.

A protein portion of a glycoprotein may be folded so as to incorporate the sugar chain portion therein in a state before denaturation. The molecular weight of such a protein portion may be, for example, 1 kDa or greater or 10 kDa or greater. The upper limit of the molecular weight of the protein portion is not particularly limited and may be, for example, 1000 kDa.

Specific examples of the glycoprotein include physiologically active substances selected from the group consisting of an antibody, a hormone, an enzyme, and a complex containing these. Here, examples of the complex include a complex of an antigen and an antibody, a complex of a hormone and a receptor, and a complex of an enzyme and a substrate. Since these glycoproteins are physiologically active substances prepared by cell culture engineering, sugar chain portions to be obtained are in a non-uniform state and the significance of shortening the time for sugar chain analysis is particularly great.

In a case where a glycoprotein contains an antibody, it is particularly important to analyze a sugar chain. In this case, it is possible to rapidly isolate a sugar chain that influences the activity or the like of the antibody. Examples of the antibody include an immunoglobulin such as IgG, IgM, IgA, IgD, or IgE; a low-molecular-weight antibody such as Fab, F(ab′), F(ab′)₂, a single-chain antibody (scFv), or a bispecific antibody (diabody); an Fc-containing molecule such as an Fc fusion protein or a peptide configured by fusion between an Fc region and another functional protein or a peptide; and a chemically modified antibody obtained by adding a chemically modifying group such as a radioactive isotope coordinating chelate or polyethylene glycol. Further, the antibody may be a monoclonal antibody or a polyclonal antibody.

Further, the antibody may be an antibody pharmaceutical candidate or an antibody pharmaceutical product. The antibody pharmaceutical candidate is a substance obtained in the process of developing an antibody pharmaceutical product and used for evaluating the activity and the safety as an antibody pharmaceutical product. In a case where a sugar chain is isolated from an antibody pharmaceutical candidate, it is possible to accelerate development of an antibody pharmaceutical product. Further, in a case where a sugar chain is isolated from an antibody pharmaceutical product, it is possible to accelerate quality management of an antibody pharmaceutical product.

<<Solid Phase>>

According to the method of the present embodiment, a glycoprotein is fixed to a solid phase. Examples of the form of fixation include a non-covalent bond (a hydrogen bond or an ionic bond) due to a specific bond; and a covalent bond. In addition, the examples do not include the form in which a glycoprotein is merely held by being applied to an electrophoretic gel or being transferred to a blotting membrane. In a case where the glycoprotein is fixed by a non-covalent bond, it is preferable that a binding rate constant ka (unit: M⁻¹s⁻¹) has an affinity of, for example, 10³ or greater, 10⁴ or greater, 10³ to 10⁵, or 10⁴ to 10⁵.

The solid phase to which a glycoprotein is fixed is not particularly limited as long as the solid phase is a carrier having a linker, which is non-covalently or covalently linked to a protein portion of a glycoprotein, on the surface thereof.

Examples of the linker included in the surface of the carrier include a ligand capable of capturing a protein portion of a glycoprotein. Examples of the ligand include a molecule (hereinafter, also simply referred to as a molecule having an affinity for a glycoprotein) having an affinity for a protein portion of a glycoprotein and a carrier in which an ion exchange group or a hydrophobic group is chemically modified in the surface.

The molecule having an affinity for a glycoprotein is not particularly limited and can be easily determined by those skilled in the art according to the glycoprotein to be captured. Examples thereof include a peptidic or proteinous ligand, an aptamer (synthetic DNA, synthetic RNA, or a peptide which can be specifically bonded to a glycoprotein), and a chemically synthetic ligand (a thiazole derivative or the like).

For example, in a case where the glycoprotein is an antibody, the molecule having an affinity for a glycoprotein may be specifically bonded to an antibody or an Fc-containing molecule which is a constant region of an antibody. More specifically, examples of the peptidic or proteinous ligand include a microorganism-derived ligand such as protein A, protein (G protein L, protein H, protein D, or protein Arp; a functional variant (analog substance) obtained by recombinant expression of these ligands; and recombinant protein such as an antibody Fc receptor. In this manner, it is possible to prepare and analyze a sugar chain sample having excellent throughput properties with respect to an antibody with a particularly high importance for sugar chain analysis.

The ion exchange group is not particularly limited as long as the ion exchange group is a functional group which is capable of capturing a glycoprotein using an ion exchange function and capable of releasing a glycoprotein by a counter ion in an ionic strength dependent manner. Preferred examples thereof include cation exchange groups such as a carboxyl group (more specifically, a carboxymethyl group or the like) and a sulfonic acid group (more specifically, a sulfoethyl group, a sulfopropyl group, or the like). As the ion exchange group, anion exchange groups such as a quaternary amino group may be used.

Examples of the hydrophobic group include an alkyl group having 2 to 8 carbon atoms and an aryl group having 2 to 8 carbon atoms. More specifically, examples thereof include a butyl group, a phenyl group, and an octyl group. These groups may be used alone or in combination of two or more kinds thereof.

In addition to those described above, the linker included in the surface of a carrier may be a linking group which is covalently bonded to a C terminal of a C terminal amino acid residue serving as a constituent element of a protein portion of a glycoprotein. Examples of such a linking group include a linking group derived from an amino group-containing compound serving as a solid phase surface modification reagent used for peptide-solid phase synthesis.

The carrier is not particularly limited as long as the carrier is a base material which is insoluble in water and is capable of fixing the above-described linker, and examples thereof include an organic carrier, an inorganic carrier, and a composite carrier. Examples of the organic carrier include synthetic polymers such as cross-linked polyvinyl alcohol, cross-linked polyacrylate, cross-linked polyacrylamide, and cross-linked polystyrene; and carriers formed of polysaccharides such as cross-linked sepharose, crystalline cellulose, cross-linked cellulose, cross-linked amylose, cross-linked agarose, and cross-linked dextran. These may be used alone or in combination of two or more kinds thereof. Examples of the inorganic carrier include glass beads, silica gel, and monolith silica.

An inorganic carrier is unlikely to contain water while an organic carrier has a property of easily containing water. According to the method of the present embodiment, since various reactions are carried out on a solid phase, it is preferable to use an inorganic carrier which is unlikely to contain water. In this manner, effects of an enzyme and/or a reagent are not reduced, which is preferable. The prevention of reduction in effects of an enzyme and/or a reagent contributes to prevention of detection for unnecessary signals during analysis. Therefore, it is preferable to use an inorganic carrier as a carrier. Further, when the carriers are inorganic carriers, for example, some carriers are not isolated by a sugar chain-isolating enzyme and elution of sugar remaining in a resin does not occur from the beginning in a case where a sugar-derived resin is used. Accordingly, appearance of unnecessary signals is easily suppressed during analysis of an isolated sugar chain.

The shape of the carrier is not particularly limited, but may be particulate or non-particulate. In a case of a particulate carrier (bead), a porous carrier may be used. In the case of a particulate carrier, the average particle diameter may be in a range of 1 to 100 μm. It is preferable that the average particle diameter is greater than or equal to the above-described lower limit from the viewpoint of liquid permeability. It is preferable that the average particle diameter is less than or equal to the above-described upper limit from the viewpoint of preventing a decrease in theoretical plate number.

Examples of the non-particulate carrier include monolith type silica gel and a membrane body. The monolith type silica gel is a bulk body of silica gel having micrometer-sized three-dimensional net-like pores (macropores) and nanometer-sized pores (mesopores). The diameter of the macropores may be in a range of 1 to 100 μm, in a range of 1 to 50 μm, in a range of 1 to 30 μm, or in a range of 1 to 20 μm. It is preferable that the diameter of the macropore is greater than or equal to the above-described lower limit from the viewpoint of liquid permeability. It is preferable that the diameter of the macropore is less than or equal to the above-described upper limit from the viewpoint of preventing a decrease in theoretical plate number. The diameter of the mesopore may be in a range of 1 to 100 nm or in a range of 1 to 50 nm. In this manner, it is possible to efficiently capture sugar.

The use volume (the volume of the carrier includes the volume of voids at the time of filling in a case of the particulate carrier and the volume of the carrier includes the volume of mesopores and macropores in a case of the non-particulate carrier) of the carrier may be in a range of 0.001 to 0.1 cm³ or in a range of 0.001 to 0.01 cm³. It is preferable that the volume is greater than or equal to the above-described lower limit from the viewpoint of preventing a decrease in theoretical plate number. It is preferable that the volume is less than or equal to the above-described upper limit from the viewpoint of liquid permeability. Further, when the volume is in the above-described range, a separate liquid after elution can be easily obtained at a concentration suitable for HPLC analysis.

The solid phase may be used in a state of filling a container such as each well of a column and a multi-well plate; each well of a filter plate; or a micro-tube.

(Preparation of Sample Containing Glycoprotein Fixed to Solid Phase)

The sample containing a glycoprotein fixed to a solid phase can be obtained by bringing a sample containing a glycoprotein into contact with the solid phase to capture the glycoprotein. In the sample containing a glycoprotein to be brought into contact with the solid phase, the glycoprotein may not be purified (separation of the glycoprotein from the impurities) from the viewpoint of rapidly performing preparation of a sugar chain. Examples of the sample include body fluid such as blood (for example, serum or plasma), lymph fluid, peritoneal exudate fluid, interstitial fluid, cerebrospinal fluid, or ascites fluid; a culture supernatant of antibody producing cells such as B cells, hybridomas, or CHO cells; and ascites fluid of animals to which antibody producing cells are transplanted. The sample may be a mixture of glycoprotein variations in which the protein portion is uniform and the sugar chain portion is non-uniform, such as a glycoprotein preparation obtained by cell culture engineering of a culture supernatant or the like.

The sample containing a protein fixed to the solid phase may be a product obtained by solid phase synthesis of a glycoprotein in addition to those described above.

The concentration of the glycoprotein in the sample containing a glycoprotein to be brought into contact with the solid phase is not particularly limited, but may be in a range of 0.1 μg/mL to 50 mg/mL. It is preferable that the concentration thereof is greater than or equal to the above-described lower limit from the viewpoint of detectability. It is preferable that the concentration thereof is less than or equal to the above-described upper limit from the viewpoint of quantitativity.

The glycoprotein to be brought into contact with the solid phase may be in a range of 0.001 μg to 100 mg or in a range of 0.001 μg to 5 mg per one container. It is preferable that the amount of glycoprotein is greater than or equal to the above-described lower limit from the viewpoint of the detection. Since the number of steps is small and the sample loss is extremely small in the method of the present embodiment, the method is particularly useful in a case where the glycoprotein has a small scale (particularly in a range of 0.001 to 500 μg). It is preferable that the amount of glycoprotein is less than or equal to the above-described upper limit from the viewpoint of quantitativity.

The sample containing the glycoprotein fixed to the solid phase may be prepared in a state in which the glycoprotein fixed to the solid phase is dispersed in a liquid component or in a state in which the liquid component is separated.

Further, the sample containing the glycoprotein fixed to the solid phase may contain impurities at the time when the capturing of the glycoprotein by bringing the sample containing the glycoprotein into contact with the solid phase is completed or when the solid phase synthesis is completed. Examples of the impurities include components contained in the sample containing the glycoprotein to be fixed to the solid phase; and a reagent used for the solid phase synthesis of the glycoprotein. More specifically, examples of the impurities include salts, low-molecular-weight compounds, proteins (proteins which are not bonded to the solid phase), and other biological molecules.

Accordingly, as the sample containing the glycoprotein fixed to the solid phase, a sample on which a cleaning treatment is performed after the capturing of the glycoprotein is completed or the solid phase synthesis is completed. In this manner, impurities can be removed while the glycoprotein is fixed to the solid phase. The cleaning can be performed by allowing the cleaning solution to permeate into the solid phase. Examples of the liquid permeation include methods of natural fall, suction, pressurization, and centrifugation.

As the cleaning solution, a solution having a composition and a liquid property, in which a bond between the protein portion of the glycoprotein and the linker in the surface of the solid phase is not cut, is appropriately selected by those skilled in the art. Specifically, a buffer solution, other aqueous solutions, and water may be used. In a case of using an aqueous solution, the pH thereof is preferably in a range of 5 to 10. In a case where the pH of the aqueous solution is in the above-described range, the activity of a sugar chain-isolating enzyme that is used in the subsequent step is easily maintained. Further, in a case where the glycoprotein is fixed to the solid phase by a non-covalent bond, the isolation of the glycoprotein is easily prevented. In a case of using a buffer solution, examples of the buffer include ammonium salts such as ammonium carbonate, ammonium bicarbonate, ammonium chloride, diammonium hydrogen citrate, and ammonium carbamate; a tris buffer such as trishydroxymethylammonium; and a phosphate.

(Container)

The sample containing the glycoprotein fixed to the solid phase is prepared in a container. It is efficient that the glycoprotein fixed to the solid phase is prepared in the container, which is preferable. The container is not particularly limited as long as the container is capable of holding a liquid and a solid phase and separating the liquid (allowing the liquid to permeate) in a state of holding the solid phase, and examples of the container include each well of a column and a multi-well plate; each well of a filter plate; and a micro-tube.

(Sugar Chain-Isolating Enzyme)

Examples of the sugar chain-isolating enzyme acting on the glycoprotein include peptide N-glycanase (PNGase F and PNGase A) and endo-β-N-acetylglucosaminidase (Endo-H, Endo-F, Endo-A, and Endo-M).

The sugar chain-isolating enzyme may be prepared in a state of being dispersed in water or a buffer solution. In a case of using a buffer solution, examples of the buffer include ammonium carbonate, ammonium bicarbonate, ammonium chloride, diammonium hydrogen citrate, and ammonium carbamate. As the buffer solution, a buffer solution having a pH of 5 to 10 is preferable. In a case where the pH of the buffer solution is in the above-described range, the activity of a sugar chain-isolating enzyme is easily maintained. Water or the buffer solution may contain a sugar chain-isolating enzyme, salts such as metal salts, and components such as a stabilizer of proteins such as glycerol.

(Deglycosylation Promoter)

The isolation step may be performed in the presence of a deglycosylation promoter. In this manner, the recovery rate of the sugar chain sample from the glycoprotein can be improved. It is preferable that the deglycosylation promoter contains an acid-derived anionic surfactant. The protein portion of the glycoprotein is denatured so that the tertiary structure is changed by the acid-derived anionic surfactant and the sugar chain-isolating enzyme easily acts on a decomposition target site. In this manner, the sugar portion is easily decomposed and isolated.

The acid-derived anionic surfactant is an anionic surfactant derived from an organic acid. Examples thereof include a carboxylic acid type anionic surfactant, a sulfonic acid type anionic surfactant, a sulfuric acid ester type anionic surfactant, and a phosphoric acid ester type anionic surfactant. Among these, a carboxylic acid type anionic surfactant is preferable. In a case where the acid-derived anionic surfactant is a carboxylic acid type anionic surfactant, it is considered that the sugar chain-isolating enzyme is unlikely to be denatured while the protein portion of the glycoprotein is denatured.

<<Acid-Derived Anionic Surfactant—Carboxylic Acid Type Anionic Surfactant>>

Examples of the carboxylic acid type anionic surfactant include a carboxylic acid and a carboxylate represented by R¹—COOX (here, R¹ represents an organic group and X represents a hydrogen atom or a cation); and an amino acid and a salt thereof (N-acylamino acid surfactant) represented by R¹CON(R²)—R³—COOX (here, R¹ represents an organic group, —N(R²)—R¹—COO— represents an amino acid residue, and X represents a hydrogen atom or a cation). Among these, an amino acid and a salt thereof (N-acylamino acid surfactant) represented by R¹CON(R²)—R³—COOX (here. R¹ represents an organic group, —N(R²)—R³—COO— represents an amino acid residue, and X represents a hydrogen atom or a cation) are preferable.

Examples of the cation X include alkali metal ions such as sodium or potassium, a triethanolamine ion, and an ammonium ion. Further, in all examples of the acid-derived anionic surfactants described below, the “salt” is intended to exemplify at least a sodium salt, a potassium salt, a triethanolamine salt, or an ammonium salt.

<<Carboxylic Acid Type Anionic Surfactant—Carboxylic Acid and Carboxylate>>

In the carboxylate represented by R¹—COOX, the organic group R¹ represents a group having at least carbon, and examples thereof include a higher alkyl group, a higher unsaturated hydrocarbon group, a hydrocarbon group having an oxyalkylene group interposed therein, and a fluorine-substituted higher alkyl group.

The number of carbon atoms of the higher alkyl group or the higher unsaturated hydrocarbon group may be in a range of 6 to 18. Specific examples of the carboxylic acid type anionic surfactant containing such a higher alkyl group or higher unsaturated hydrocarbon group include an octanoate, a decanoate, a laurate, a myristate, a palmitate, a stearate, an oleate, and a linoleate. Further, the above-described higher alkyl group or higher unsaturated hydrocarbon group may be substituted, and the substituent may be an alkyl group or alkoxycarbonyl group having 1 to 30 carbon atoms.

In the hydrocarbon group having an oxyalkylene group interposed therein, one or more oxyalkylene groups may be included in the main chain thereof. Examples of the oxyalkylene group include an oxyethylene group, an oxy-n-propylene group, and an oxyisopropylene group. As the hydrocarbon group having an oxyalkylene group interposed therein, a group represented by R⁴—(CH₂CH₂)ₙ—R⁵— may be exemplified.

Here, R⁴ may represent a higher alkyl group, a higher unsaturated hydrocarbon group, or a substituted or unsubstituted aryl group. The number of carbon atoms of the higher alkyl group or the higher unsaturated hydrocarbon group may be in a range of 6 to 18. Examples of the aryl group include a phenyl group and a naphthyl group. In a case of a substituted aryl group, the substituent may be a linear or branched alkyl group, and the number of carbon atoms of the linear or branched alkyl group may be in a range of 1 to 30. Particularly in a case of a phenyl group, the substituent may be substituted at the para position with respect to a sulfonyl group. Further, n may represent a number of 1 to 10. R⁵ may represent a sigma bond or an alkylene group such as an ethylene group, a methylene group, or an n-propylene group. Specific examples of such a carboxylate include a laureth carboxylate (such as laureth-4-carboxylate or laureth-6-carboxylate) and a trideceth carboxylate (such as trideceth-4-carboxylate or trideceth-6-carboxylate).

In the fluorine-substituted higher alkyl group, one or more hydrogen atoms are substituted with a fluorine atom. The fluorine-substituted higher alkyl group may be perfluoroalkyl group in which all hydrogen atoms are substituted with fluorine. Further, the carbon atoms thereof may be in a range of 6 to 18. Specific examples of the perfluoroalkylcarboxylic acid and the perfluoroalkylcarboxylate include perfluorooctanoic acid, perfluorononanoic acid, perfluorooctanoate, and perfluornonanoate.

<<Carboxylic Acid Type Anionic Surfactant—Amino Acid and Salt Thereof>>

In the amino acid and the salt thereof represented by R¹CON(R²)—R³—COOX, the organic group R¹ and the cation X have the same definitions as those for the organic group R¹ and the cation X in the carboxylic acid or the carboxylate described above.

Further. R² represents a hydrogen atom or an alkyl group (such as a methyl group, an ethyl group, an n-propyl group, or an isopropyl group). R³ may represent a substituted or unsubstituted ethylene group, methylene group, or n-propylene group and may form a ring together with nitrogen atoms on the N-terminal side. Accordingly, the amino acid residue represented by —N(R²)—R³—COO— may be an α-amino acid residue, a β-amino acid residue, a γ-amino acid residue, a residue derived from a natural amino acid, or a residue derived from an unnatural amino acid. Examples thereof include residues derived from an amino acid such as a sarcosine residue, a glutamic acid residue, a glycine residue, an aspartic acid residue, a proline residue, and a β-alanine residue.

Specific examples of the amino acid or the salt thereof (that is, a N-acylamino acid surfactant) in a case where R² represents a hydrogen atom include N-lauroyl aspartate, N-lauroyl glutamic acid, N-lauroyl glutamate, N-myristoyl glutamate, N-cocoylalanine salt, N-cocoylglycine salt. N-cocoyl glutamate. N-palmitoyl glutamate. N-palmitoyl proline, N-palmitoyl proline salt, N-undecylenoyl glycine, N-laudecylenoyl glycine salt, and N-stearoyl glutamine salt. In a case where the acid-derived anionic surfactant is a N-acylamino acid surfactant, there is a tendency that the protein portion of the glycoprotein is easily denatured and the sugar chain-isolating enzyme is unlikely to be denatured.

Specific examples of the amino acid or the salt thereof in a case where R² represents an alkyl group (in other words, a N-acyl-N-alkylamino acid surfactant) include N-cocoyl-N-methylalanine, N-cocoyl-N-methylalanine salt, N-myristoyl-N-methyl-β-alanine. N-myristoyl-N-methyl-β-alanine salt, N-myristoyl sarcosine salt, N-lauroyl-N-methylalanine, N-lauroyl-N-methylalanine salt, N-lauroyl-N-ethylglycine, N-lauroyl-N-isopropylglycine salt, N-lauroyl-N-methyl-β-alanine, N-lauroyl-N-methyl-β-alanine salt. N-lauroyl-N-ethyl-β-alanine, N-lauroyl-N-ethyl-β-alanine salt, N-lauroyl sarcosine, N-lauroyl sarcosine salt, N-cocoyl sarcosine, N-cocoyl sarcosine salt, N-oleoyl-N-methyl-β-alanine, N-oleoyl-N-methyl-β-alanine salt, N-oleoyl sarcosine, N-oleoyl sarcosine salt, N-linoleyl-N-methyl-β-alanine, N-palmitoyl-N-methyl-β-alanine, and N-palmitoyl sarcosine salt. In a case where the acid-derived anionic surfactant is a N-acyl-N-alkylamino acid surfactant, there is a tendency that the protein portion of the glycoprotein is more easily denatured and the sugar chain-isolating enzyme is unlikely to be denatured.

<<Acid-Derived Anionic Surfactant—Sulfonic Acid Type Anionic Surfactant>>

The sulfonic acid type anionic surfactant is a sulfonic acid or a sulfonate represented by R¹—SO₃X (here, R¹ represents an organic group and X represents a hydrogen atom or a cation). The organic group R¹ represents a group having at least carbon, and examples of the organic group include a higher alkyl group, a higher unsaturated hydrocarbon group, a hydrocarbon group having an oxyalkylene group interposed therein, a fluorine-substituted higher alkyl group, a substituted or unsubstituted aryl group, and a higher alkyl group or higher unsaturated hydrocarbon group having a divalent linking group (such as —O—, —CO—, —CONH—, or —NH—) interposed therein.

The higher alkyl group, the higher unsaturated hydrocarbon group, the hydrocarbon group having an oxyalkylene group interposed therein, the fluorine-substituted higher alkyl group, and the cation X among the examples of the organic group R¹ have the same definitions as those for the organic group R¹ and the cation X in the carboxylic acid or the carboxylate.

Specific examples thereof include 1-hexanesulfonate, 1-octanesulfonate, l-decanesulfonate, and 1-dodecanesulfonate; perfluorobutanesulfonate, perfluorobutanesulfonate, perfluorooctanesulfonate, and perfluorooctanesulfonate; tetradecenesulfonate; and an alpha sulfo fatty acid methyl ester salt (CH₃(CH₂)ₙCH(SO₃X)COOCH₃) (n represents an integer of 1 to 30).

In a case where the organic group R¹ represents a substituted or unsubstituted aryl group, examples of the aryl group include a phenyl group and a naphthyl group. In a case of a substituted aryl group, the substituent may be a linear or branched alkyl group and the number of carbon atoms of the linear or branched alkyl group may be in a range of 1 to 30. Particularly in a case of a phenyl group, the substituent may be substituted at the para position with respect to a sulfonyl group. Examples of such an aromatic sulfonate include a toluene sulfonate, a cumene sulfonate, an octyl benzene sulfonate, a dodecyl benzene sulfonate, a naphthalene sulfonate, a naphthalene disulfonate, a naphthalene trisulfonate, and a butyl naphthalene sulfonate.

Examples of the sulfonic acid type surfactant in a case where the organic group R¹ represents a higher alkyl group or higher unsaturated hydrocarbon group having a divalent linking group (such as —O—, —CO—, —CONH—, or —NH—) interposed therein include an isethionate which is O-substituted with the higher alkyl group or higher unsaturated hydrocarbon group; and a taurine salt which is N-substituted with the higher alkyl group or higher unsaturated hydrocarbon group. The number of carbon atoms of the higher alkyl group or higher unsaturated hydrocarbon group may be in a range of 6 to 18. Specific examples of such a sulfonic acid type surfactant include a cocoyl isethionate, a cocoyl taurine salt, cocoyl-N-methyl taurine, N-oleoyl-N-methyl taurine salt, N-stearoyl-N-methyl taurine salt, and N-lauroyl-N-methyl taurine salt.

<<Acid-Derived Anionic Surfactant—Sulfuric Acid Ester Type Anionic Surfactant>>

The sulfuric acid ester type anionic surfactant is a sulfuric acid ester salt represented by R¹—OSO₃X (here, R¹ represents an organic group and X represents a cation). The organic group R^{r} represents a group having at least carbon, and examples thereof include a higher alkyl group, a higher unsaturated hydrocarbon group, a hydrocarbon group having an oxyalkylene group interposed therein, and a fluorine-substituted higher alkyl group. These groups have the same definitions as those for R¹ in the carboxylic acid type surfactant described above. Examples of the cation X include an alkali metal ion such as sodium or potassium, a triethanolamine ion, and an ammonium ion.

Specific examples of the sulfuric acid ester salt include a lauryl sulfate, a myristyl sulfate, a laureth sulfate (C₁₂H₂₅(CH₂CHO₂)ₙOSO₃X, here, n represents an integer of 1 to 30), and sodium polyoxyethylene alkyl phenol sulfonate (C₈H₁₇CH₄O[CH₂CH₂O]₃SO₃X).

<<Acid-Derived Anionic Surfactant—Phosphoric Acid Ester Type Anionic Surfactant>>

The phosphoric acid ester type anionic surfactant is a phosphoric acid ester or a phosphoric acid ester salt represented by R¹—OSO₃X (here, R¹ represents an organic group and X represents a hydrogen atom or a cation). The organic group R¹ represents a group having at least carbon, and examples thereof include a higher alkyl group, a higher unsaturated hydrocarbon group, a hydrocarbon group having an oxyalkylene group interposed therein, and a fluorine-substituted higher alkyl group. These groups have the same definitions as those for R¹ in the carboxylic acid type surfactant described above. Examples of the cation X include an alkali metal ion such as sodium or potassium, a triethanolamine ion, and an ammonium ion.

Specific examples of the phosphoric acid ester or phosphoric acid ester salt include lauryl phosphoric acid and lauryl phosphate.

<<Composition of Deglycosylation Promoter>>

The deglycosylation promoter may be prepared in a state in which the acid-derived anionic surfactant is dissolved or dispersed in water or a buffer solution. In a case of using a buffer solution, examples of the buffer include ammonium salts such as ammonium carbonate, ammonium bicarbonate, ammonium chloride, diammonium hydrogen citrate, and ammonium carbamate; a tris buffer such as trishydroxymethylammonium; and a phosphate. As the buffer solution, a buffer solution having a pH of 5 to 10 is preferable. In a case where the pH of the buffer solution is in the above-described range, the activity of a sugar chain-isolating enzyme is easily maintained. Examples of components other than the acid-derived anionic surfactant contained in water or the buffer solution in the deglycosylation promoter include salts such as metal salts other than surfactants.

(Operation and Reaction Condition of Isolation Step)

In the isolation step, an isolating reaction solution containing a glycoprotein and a sugar chain-isolating enzyme, in which the optimum conditions (the temperature and the pH) for the sugar chain-isolating enzyme are satisfied may be prepared.

In a case of using a deglycosylation promoter, an isolating reaction solution in which the optimum conditions (the temperature, the pH, and the like) of the sugar chain-isolating enzyme are satisfied and which contains a glycoprotein, an acid-derived anionic surfactant, and a sugar chain-isolating enzyme may be prepared. Therefore, in the case of using a deglycosylation promoter, a sample containing a fixed glycoprotein (hereinafter, also simply referred to as a sample containing a glycoprotein), a deglycosylation promoter, and a sugar chain-isolating enzyme may be mixed in any operation procedure.

For example, the sample containing the glycoprotein, the deglycosylation promoter, and the sugar chain-isolating enzyme were mixed with each other at the same timing to prepare an isolating reaction solution. Further, the isolating reaction solution may be prepared by adding the deglycosylation promoter and then adding the sugar chain-isolating enzyme. Further, in a case where the glycoprotein fixed to the solid phase is obtained by performing a pre-treatment described below and the deglycosylation promoter and the surfactant used for the pre-treatment are formed of the same substance, the surfactant in an amount corresponding to the amount of the deglycosylation promoter is added to the surfactant in an amount corresponding to the amount of a pre-treatment agent and then added in advance during the pre-treatment, and then only the sugar chain-isolating enzyme may be added during the isolation step (because of the state in which the deglycosylation promoter is already present).

Specifically, the isolating reaction solution into which all components are mixed is prepared, the optimum temperature are set, and then a reaction of isolating the sugar chain from the glycoprotein can be carried out. In this case, the reaction time may be in a range of 5 seconds to 24 hours.

In a case of using the deglycosylation promoter, the sample containing the glycoprotein and the acid-derived anionic surfactant may be mixed with each other in advance so that the protein portion of the glycoprotein is denatured and then the mixture may be mixed with the sugar chain-isolating enzyme. In this case, the denaturation time may be in a range of 5 seconds to 24 hours and the sugar chain-isolation time may be in a range of 5 seconds to 24 hours.

In the isolating reaction solution, the concentration of the glycoprotein may be in a range of 0.1 μg/mL to 100 mg/mL or in a range of 1 μg/mL to 10 mg/mL. It is preferable that the concentration of the glycoprotein in the isolating reaction solution is greater than or equal to the above-described lower limit from the viewpoint of detectability. It is preferable that the concentration thereof is less than or equal to the above-described upper limit from the viewpoint of quantitativity.

In a case of using the deglycosylation promoter, the concentration of the acid-derived anionic surfactant in the isolating reaction solution may be in a range of 0.01% to 30% by mass, in a range of 0.2% to 1.0% by mass, in a range of 0.2% to 0.3% by mass, or in a range of 0.22% to 0.27% by mass. Alternatively, the amount of the acid-derived anionic surfactant may be set to be in a range of 0.001 μg to 100 mg with respect to 1 μg of the glycoprotein.

By setting the amount of the acid-derived anionic surfactant to be used to be in the above-described range, the activity of the sugar chain-isolating enzyme is maintained, the recovery amount of the isolated sugar chain becomes excellent, and the stability of the recovery amount also becomes excellent. Further, it is preferable that the purification of the isolated sugar chain is performed by a solid phase carrier from the viewpoint of preventing the drying time from being excessively long.

The concentration of the sugar chain-isolating enzyme in the isolating reaction solution may be in a range of 0.001 μU/mL to 1000 mU/ml, or in a range of 0.01 μU/mL 15 to 100 mU/mL. Alternatively, the amount of the sugar chain-isolating enzyme may be set to be in a range of 0.001 μU to 1000 mU with respect to 1 μg of the glycoprotein. In a case where the amount of the sugar chain-isolating enzyme to be used is in the above-described range, the sugar chain can be efficiently isolated.

The reaction pH may be adjusted to the optimum pH of the sugar chain-isolating enzyme and may be in a range of 5 to 10. The reaction temperature may be adjusted to the optimum temperature of the sugar chain-isolating enzyme and may be in a range of 4° C. to 90° C.

The reaction time varies depending on the scale or the like of the glycoprotein and may be in a range of 5 seconds to 24 hours. It is preferable that the reaction system of the isolation step is made to be an open system and heated so that the solvent is evaporated. The heating temperature may be 40° C. or higher or 45° C. or higher. In this manner, since the solvent during the isolation step is evaporated and the concentration of the reaction solution gradually increases, it is easy to set the concentration of the reaction solution to the extent that the isolation of the sugar chain efficiently proceeds regardless of the scale of the glycoprotein provided for the method of the present embodiment. Further, since solvent removal is performed together with the isolation reaction, the time for performing the solvent removal step separately from the isolation step is shortened or becomes unnecessary so that it becomes possible to rapidly prepare a sugar chain. The upper limit of the heating temperature may be, for example, 80° C. from the viewpoint of preventing denaturation of the sugar chain-isolating enzyme.

(Isolated Product)

The isolated product obtained by the isolation step contains the isolated sugar chain and the protein bonded to the solid phase. In the protein bonded to the solid phase, the peptide bond between amino acid residues in the protein portion constituting the glycoprotein is not cut. The isolated product may be obtained in a state of containing a solvent or in a state of an evaporated and dried product in which the solvent is completely evaporated in a case where an open system and heating conditions are provided particularly in the isolation step.

According to the method of the present embodiment, since the protein portion is in a state being fixed to the solid phase while the sugar chain is isolated, the protein portion is removed only by separating the solid phase. Meanwhile, the separate liquid obtained by separating the solid phase is a mixed solution in which the surfactant used in the pre-treatment step and the deglycosylation promoter used in the isolation step together with the isolated sugar chain coexist. Depending on the method of analyzing the sugar chain, the sugar chain may be provided for the analysis in a state of a mixed solution in which the sugar chain, the above-described surfactant, and the like coexist in some cases, but it is preferable to purify the sugar chain from the mixed solution before the analysis in a case where the analysis is carried out by means of mass analysis or the like.

In a case of purifying the sugar chain, for example, the mixed solution can be brought into contact with a solid phase carrier for purification using a polymer containing a hydrazide group as a solid phase carrier for purification. In the mixed solution, the isolated sugar chain is in an equilibrium state between a cyclic hemiacetal type and an acyclic aldehyde type, the aldehyde group-CHO specifically reacts with the hydrazide group-NH—NH₂ to form a stable bond —C═N—NH—. In this manner, the isolated sugar chain can be captured by the solid phase carrier for purification.

The sugar chain captured by the solid phase carrier for purification may be re-isolated. As a re-isolation method, a method of bringing a mixed solvent of an acid and an organic solvent or a mixed solvent of an acid, water, and an organic solvent into contact with the solid phase carrier to cause a reaction may be exemplified. The pH of the mixed solvent may be in a range of 2 to 9, in a range of 2 to 7, or in a range of 2 to 6. It is preferable that the reaction is carried out in the vicinity of neutrality from weak acidity from the viewpoint of suppressing hydrolysis of the sugar chain such as releasing a sialic acid residue. However, strong acid conditions with a low pH are also accepted.

As described below, the isolated sugar chain can be modified by a low-molecular-weight compound (labeled compound). The low-molecular-weight compound can be appropriately selected according to the analysis method. Further, the low-molecular-weight compound is distinguished from a polymer compound constituting the solid phase carrier, and it is preferable that the low-molecular-weight compound is a compound which can be dissolved in water, a buffer solution, or an organic solvent.

[Pre-Treatment Step]

The method of the present embodiment may further include a pre-treatment step before the isolation step. In this manner, the sugar chain can be easily isolated from the glycoprotein without performing a decomposition treatment on the protein portion. As the result, the time taken for the sugar chain-isolating treatment can be greatly shortened.

In the pre-treatment step, a pre-treatment agent containing a surfactant is brought into contact with the sample containing the glycoprotein fixed to the solid phase. The pre-treatment step may be performed after the capturing of the glycoprotein is completed by bringing the sample containing the glycoprotein into contact with the solid phase, after the solid phase synthesis is completed, or after the cleaning treatment is performed and before the glycoprotein is brought into contact with the sugar chain-isolating enzyme. By performing the pre-treatment step, the sugar chain-isolating enzyme easily acts on the glycoprotein in the isolation step.

The surfactant contained in the pre-treatment agent may be any of an anionic surfactant, a cationic surfactant, an amphoteric surfactant, and a non-ionic surfactant.

The anionic surfactant is not particularly limited, and examples thereof include a salt of fatty acid such as soap, an alkyl benzene sulfonate, a higher alcohol sulfuric acid ester salt, a polyoxyethylene alkyl ether sulfate, α-sulfofatty acid ester, α-olefin sulfonate, a monoalkyl phosphoric acid ester salt, and an alkyl sulfonate. However, it is preferable that the anionic surfactant is an anionic surfactant which can be used as a deglycosylation promoter used in the sugar chain isolation step described below (in the present specification, the anionic surfactant which can be used as a deglycosylation promoter is particularly referred to as an acid-derived anionic surfactant). In a case where the acid-derived anionic surfactant is used in the pre-treatment step, the surfactant may be a surfactant which is the same as or different from the surfactant exemplified as a deglycosylation promoter used in the sugar chain isolation step.

The cationic surfactant is not particularly limited, and examples thereof include an alkyl trimethyl ammonium salt, a dialkyl dimethyl ammonium salt, an alkyl dimethyl benzyl ammonium salt, and an amine salt. The amphoteric surfactant is not particularly limited, and examples thereof include an alkylamino fatty acid salt, alkyl betaine, and alkylamine oxide. The non-ionic surfactant is not particularly limited, and examples thereof include polyoxyethylene alkyl ether, polyoxyethylene alkyl phenyl ether, alkyl glucoside, polyoxyethylene fatty acid ester, sucrose fatty acid ester, sorbitan fatty acid ester, polyoxyethylene sorbitan fatty acid ester, fatty acid alkanolamide, and a polyoxyethylene-polyoxypropylene block copolymer.

The pre-treatment agent may be used in a state in which the surfactant is dissolved in water or a buffer solution. In a case of using a buffer solution, examples of the buffer include ammonium salts such as ammonium carbonate, ammonium bicarbonate, ammonium chloride, diammonium hydrogen citrate, and ammonium carbamate; a tris buffer such as trishydroxymethylammonium; and a phosphate. As the buffer solution, a buffer solution having a pH of 5 to 10 is preferable. In a case where the pH of the buffer solution is in the above-described range, the activity of a sugar chain-isolating enzyme used in the subsequent step is easily maintained. In the sample containing the glycoprotein, examples of components other than the glycoprotein contained in water or the buffer solution include salts such as metal salts, and a stabilizer of proteins such as glycerol.

The concentration of the surfactant in the pre-treatment agent may be in a range of 0.01% to 30% by mass, in a range of 0.2% to 1.0% by mass, in a range of 0.2% to 0.3% by mass, or in a range of 0.22% to 0.27% by mass. In a case where the concentration thereof is greater than or equal to the above-described lower limit or the concentration thereof is less than or equal to the above-described upper limit, the sugar chain isolated in the subsequent sugar chain isolation step can be obtained at a high recovery rate.

The pre-treatment agent is separated from the glycoprotein fixed to the solid phase after being brought into contact with the solid phase. The separation may be performed at once after all of a predetermined amount of pre-treatment agent to be used is put into a container or the separation may be performed whenever a part of the predetermined amount of pre-treatment agent is put into the container several times. The separation of the pre-treatment agent can be performed by reducing the pressure, performing centrifugation, or the like.

The glycoprotein fixed to the solid phase on which the pre-treatment step has been performed can be provided for the isolation step described below without performing cleaning from the viewpoint of rapid preparation. However, a cleaning operation may be performed after the pre-treatment step and before the isolation step.

[Labeling Step]

The labeling step is performed using the same container as the container used in the isolation step. Therefore, in the labeling step, a labeling reagent (labeling reaction solution) containing a labeled compound is added to the isolated product in the container used in the isolation step to obtain a labeled product containing a labeled substance of the sugar chain.

(Labeled Compound)

The labeled compound is not particularly limited as long as the labeled compound contains a reactive group with respect to a sugar chain and a modifying group to be attached to the sugar chain. Examples of the reactive group with respect to a sugar chain include an oxylamino group, a hydrazide group, and an amino group. The modifying group can be appropriately selected by those skilled in the art according to the method of analyzing a sugar chain.

For example, in a case where the labeled compound includes an oxylamino group or a hydrazide group as a reactive group with respect to a sugar chain, as the modifying group to be attached to the sugar chain, an amino acid residue selected from the group consisting of an arginine residue, a tryptophan residue, a phenylalanine residue, a tyrosine residue, a cysteine residue, and a lysine residue can be selected.

It is preferable that the labeled compound contains an arginine residue from the viewpoints of promoting ionization at the time of MALDI-TOF-MS measurement of the modified sugar chain and improving the detection sensitivity. It is preferable that the labeled compound contains a tryptophan residue from the viewpoints of improving the separability and improving the fluorescence detection sensitivity at the time of reversed phase HPLC detection of the modified sugar chain because the tryptophan residue is fluorescent and hydrophobic. It is preferable that the labeled compound contains a phenylalanine residue and/or a tyrosine residue from the viewpoint of being suitable for detection using U V absorption of the modified sugar chain. In a case where the labeled compound contains a cysteine residue, labeling with a labeling reagent such as an ICAT reagent (Applied Biosystems, USA) can be made using a —SH group of the residue as a target. In a case where the labeled compound contains a lysine residue, labeling with a labeling reagent such as an iTRAQ reagent (Applied Biosystems, USA) and an ExacTag reagent (Perkin Inc., USA) can be made using an amino group of the residue as a target. In a case where the labeled compound contains a tryptophan residue, labeling with an NBS reagent (Shimadzu Corporation, Japan) can be made using an indole group of the residue as a target.

For example, in a case where the labeled compound contains an amino group as a reactive group with respect to the sugar chain, an aromatic group may be exemplified as a modifying group to be attached to the sugar chain. When the labeled compound containing an amino group and an aromatic group is used, modification is performed by reductive amination. Since the aromatic group has UV-visible absorption characteristics or fluorescence characteristics, the detection sensitivity at the time of UV detection or fluorescence detection is improved, which is preferable.

Specific examples of the labeled compound that provides such an aromatic group include 8-aminopyrene-1,3,6-trisulfonate, 8-aminonaphthalene-1,3,6-trisulphonate, 7-amino-1,3-naphtalenedisulfonic acid, 2-amino9(10 OH)-acridone, 5-aminofluorescein.dansylethylenediamine, 2-aminopyridine, 7-amino-4-methylcoumarine, 2-aminobenzamide, 2-aminobenzoic acid, 3-aminobenzoic acid, 7-amino-1-naphthol, 3-(acetylamino)-6-aminoacridine, 2-amino-6-cyanoethylpyridine, ethyl p-aminobenzoate, p-aminobenzonitrile, and 7-aminonaphthalene-1,3-disulfonic acid.

Among these, 2-aminobenzamide is preferable from the viewpoint that 2-aminobenzamide is relatively unlikely to be affected by impurities (for example, salts, proteins, and other biological molecules) even in a case where the reaction scale is large. In addition, the method of the present embodiment is particularly useful in a case where the reaction scale is small. Since 2-aminobenzamide is unlikely to be affected by impurities as the reaction scale is small, 2-aminobenzamide can be applied to various labeling reagents (labeling reaction solutions). Further, derivatives of the above-described compounds are also preferably used as long as the functions of the labeled compound are maintained.

The labeled compound is used by being dissolved in water, a buffer solution, and/or an organic solvent. Examples of the buffer solution include the aqueous solutions of the buffers which are the same as those used in the isolation step. Examples of the organic solvent include a polar organic solvent such as N-methylpyrrolidone (NMP), dimethylsulfoxide (DMSO), or acetic acid; and a non-polar solvent such as hexane.

In the modification performed by reductive amination, efficient labeling can be carried out by allowing an aldehyde group formed at a reducing terminal of the sugar chain to react with an amino group of the labeled compound and reducing the formed Schiff base using a reducing agent so that a modifying group is introduced into the reducing terminal of the sugar chain.

Examples of the reducing agent include sodium cyanoborohydride, sodium triacetoxyborohydride, methylamine borane, dimethylamine borane, trimethylamine borane, picoline borane, and pyridine borane.

Among these, from the viewpoints of safety and reactivity, it is preferable to use picoline borane (2-picoline-borane). From the same viewpoint, in a case where picoline borane is used as a reducing agent, it is preferable to use 2-aminobenzamide as the labeled compound.

(Operation and Reaction Conditions of Labeling Step)

In the labeling step, the labeling reagent (labeling reaction solution) is added to the isolated product. In a case where modification is performed by reductive amnination, the labeling reagent may contain a labeled compound containing an amino group and an aromatic group, a reducing agent, and a solvent. In the labeling step, the container used in the isolation step is continuously used, and a treatment of changing the relative composition (the ratio of components other than the solvent), such as cleaning of the isolated product, is not performed while the labeling reagent is added. Further, addition of water, a buffer solution, and/or an organic solvent to the isolated product such that the isolated product is dissolved or diluted is accepted.

A labeling reaction system is constructed in a state in which the labeling reaction solution containing the sugar chain and the labeled compound in a solvent, using water, a buffer solution, and/or an organic solvent as a solvent, is mixed with the protein fixed to the solid phase and residues from the isolation step.

Examples of the buffer solution include the aqueous solutions of the buffers which are the same as those used in the isolation step described above. Examples of the organic solvent include an aprotic polar organic solvent such as dimethyl sulfoxide (DMSO), dimethylformamide (DMF), or N-methylpyrrolidone (NMP); a protic polar organic solvent such as an organic acid (formic acid, acetic acid, propionic acid, or butyric acid) or alcohol (methanol, ethanol, or propanol); and an aprotic non-polar solvent such as hexane. These solvents may be used alone or in combination of two or more kinds thereof.

In the labeling reaction solution, the labeling reagent (labeling reaction solution) may be used at 0.1 to 10 times by volume or at 0.5 to 5 times by volume of the use volume of the carrier. In addition, the concentration of the labeled compound in the labeling reagent may be in a range of 1 to 20 M or in a range of 2 to 15 M. It is preferable that the amount of the labeled compound is greater than or equal to the above-described lower limit front the viewpoint that the labeling is performed quantitatively. Further, it is preferable that the amount thereof is less than or equal to the above-described upper limit from the viewpoint of easily removing the excess reagent.

The concentration of the reducing agent in the labeling reaction solution may be in a range of 0.5 to 10 M or in a range of 1 to 7.5 M. It is preferable that the amount of the reducing agent is greater than or equal to the above-described lower limit from the viewpoint that the labeling is performed quantitatively. Further, it is preferable that the amount thereof is less than or equal to the above-described upper limit from the viewpoint of easily removing the excess reagent.

The solvent may be used in an amount of 0.5 to 10 times by volume or 1 to 5 times by volume of the use volume of the carrier. It is preferable that the amount of the solvent is greater than or equal to the above-described lower limit from the viewpoint of solubility. Further, it is preferable that the amount thereof is less than or equal to the above-described upper limit from the viewpoint that the labeling is performed quantitatively.

The reaction temperature of the labeling reaction solution may be in a range of 4° C. to 80° C. or in a range of 25° C. to 70° C. It is preferable that the reaction temperature is higher than or equal to the above-described lower limit from the viewpoint of shortening the reaction time. Further, it is preferable that the reaction temperature is lower than or equal to the above-described upper limit from the viewpoint of suppressing partial decomposition of the sugar chain due to a high temperature. The reaction time for the labeling reaction solution may be in a range of 5 to 600 minutes or in a range of 30 to 300 minutes. It is preferable that the reaction time is longer than or equal to the above-described lower limit from the viewpoint of quantitative labeling. Further, it is preferable that the reaction time is shorter than or equal to the above-described upper limit from the viewpoint of suppressing partial decomposition of the sugar chain.

Since the labeling reaction rapidly proceeds at room temperature, a sugar chain-labeled substance is generated due to an action of the labeled compound from when the labeling reagent (labeling reaction solution) is added. Therefore, a separation step described below can be performed at an arbitrary timing after the labeling reagent is added regardless of whether the reaction has been completed. Alternatively, a separate liquid is obtained by performing the separation step described below after the isolation step and then the labeling reagent may be added to the separate liquid.

Hereinafter, a case where picoline borane is used as a reducing agent will be described. In the case where picoline borane is used as a reducing agent, it is preferable that a protic solvent is included as a solvent. In this manner, since the labeled compound (preferably 2-aminobenzamide, the same applies hereinafter in a case of using picoline borane) and picoline borane can be dissolved at a high concentration, the time required for the labeling step is shortened.

That is, the labeling reagent (labeling reaction solution) may contain 2-aminobenzamide, picoline borane, and a solvent. By using picoline borane with low toxicity, labeling with high safety can be performed.

From the viewpoint of more preferably obtaining the effects of shortening the time required for the labeling step, it is preferable that the protic solvent is an organic acid such as formic acid, acetic acid, propionic acid, or butyric acid. Further, it is preferable the organic acid is a liquid in the labeling reaction system. From the viewpoint of ease of an operation, it is preferable that the organic acid is acetic acid.

The concentration of the protic solvent in the solvent may be in a range of 40% to 100% by volume. In this manner, excellent labeling efficiency is obtained. From the viewpoint of obtaining more excellent labeling efficiency, the concentration of the protic solvent in the solvent may be in a range of 50% to 100% by volume may be in a range of 75% to 100% by volume.

In a case where the boiling point of the above-described protic solvent is relatively low (for example, in a case where the boiling point thereof is lower than 140° C.), a solvent having a boiling point higher than that of the protic solvent may be used in combination with the protic solvent. In this manner, the volatilization rate of the protic solvent having a relatively low boiling point in the labeling step. As the result, undesired precipitation of an unreacted substance can be suppressed during the labeling step. In this manner, the labeled sugar chain can be obtained with excellent yield. The form of using a solvent having a high boiling point (hereinafter, a high-boiling point solvent) together can be selected in a case where the scale of the sugar chain is small, a case where the amount of solvent is small, and/or a case where the reaction time becomes longer.

As the above-described high-boiling point solvent, for example, an aprotic solvent having a boiling point of 140° C. to 200° C. may be used. Specific examples of the high-boiling point solvent include dimethylsulfoxide, dimethylfonnamide, and N-methylpyrrolidone.

In a case where a high-boiling point solvent is used together, the high-boiling point solvent is preferably used in an amount of lower % by volume than that of the protic solvent, may be greater than or equal to 4% by volume and less than 100% by volume of the protic solvent, or may be 4% to 70% by volume of the protic solvent, from the viewpoints of improving the solubility and reactivity of 2-aminobenzamide serving as the labeled compound and the reducing agent. It is preferable that the amount of the high-boiling point solvent is greater than or equal to the above-described lower limit from the viewpoint of decreasing the volatilization rate of the protic solvent. Further, it is preferable that the amount thereof is less than or equal to the above-described upper limit from the viewpoints of easily obtaining the effects of the protic solvent (effects of improving the solubility and reactivity of 2-aminobenzamide serving as the labeled compound and the reducing agent).

In a case where picoline borane is used as a reducing agent, it is most preferable to use a mixed solvent of acetic acid and dimethylsulfoxide as a solvent.

In a case where picoline borane is used as a reducing agent, the concentration of the labeled compound of the labeling reagent (labeling reaction solution) may be in a range of 1 to 20 M or in a range of 2 to 15 M. It is preferable that the concentration of the labeled compound is greater than or equal to the above-described lower limit from the viewpoint of shortening the time for the labeling step. Further, it is preferable that the concentration thereof is less than or equal to the above-described upper limit from the viewpoint of easily removing the excess reagent.

The amount of the picoline borane in the labeling reaction solution may be in a range of 0.5 to 10 M or in a range of 1 to 7.5 M. It is preferable that the amount of the picoline borane is greater than or equal to the above-described lower limit from the viewpoint of shortening the time for the labeling step. Further, it is preferable that the amount thereof is less than or equal to the above-described upper limit from the viewpoint of easily removing the excess reagent.

In a case where picoline borane is used as a reducing agent, the amount of the solvent may be in a range of 0.1 to 10 times by volume or in a range of 0.5 to 5 times by volume of the use volume of the carrier. It is preferable that the amount of the solvent is greater than or equal to the above-described lower limit from the viewpoint of solubility. Further, it is preferable that the amount thereof is less than or equal to the above-described upper limit from the viewpoint of shortening the time for the labeling step.

The reaction temperature of the labeling reaction solution may be in a range of 4° C. to 80° C. or in a range of 25° C. to 70° C. It is preferable that the reaction temperature is greater than or equal to the above-described lower limit from the viewpoint of shortening the reaction time. Further, it is preferable that the reaction temperature is less than or equal to the above-described upper limit from the viewpoint of suppressing partial decomposition of the sugar chain due to a high temperature. The reaction time for the labeling reaction solution may be in a range of 2 to 120 minutes or in a range of 5 to 40 minutes. It is preferable that the reaction time is longer than or equal to the above-described lower limit from the viewpoint of quantitative labeling. Further, it is preferable that the reaction time is shorter than or equal to the above-described upper limit from the viewpoint of suppressing partial decomposition of the sugar chain.

(Labeled Product)

A labeled substance of the sugar chain and the protein bonded to the solid phase are present in the container after the labeling step. Therefore, the labeled product obtained by the labeling step may contain the labeled substance of the sugar chain and the protein bonded to the solid phase. In the protein bonded to the solid phase, the peptide bond between amino acid residues in the protein portion constituting the glycoprotein is not still cut. The labeled product may be contained in water, the buffer solution, and/or the organic solvent.

[Separation Step]

(Elution of Sugar Chain-Labeled Substance)

After the labeling step, a separation step of performing solid-liquid separation from the labeled product to obtain a separate liquid which contains a labeled substance of the sugar chain may be performed. In this manner, the labeled substance of the sugar chain can be easily separated. For example, the labeled substance of the sugar chain can be eluted by liquid permeation of the eluent through the labeled product. The eluent used in this case may be a water-based solution such as water, an aqueous solution, or a colloidal solution. As the eluent, a solution having a property of cutting ability with respect to the bond between the solid phase and the protein portion may be selected (in a case where the labeled sugar chain is analyzed by chromatography) or a solution which does not have such a property may be selected (a case where the labeled sugar chain is analyzed by mass analysis). In this manner, a separate liquid containing a labeled substance of the sugar chain is obtained.

In a case where the excess labeled compound used in the labeling step and the deglycosylation promoter used in the isolation step are used together with the labeled substance of the sugar chain, unnecessary substances of the acid-derived anionic surfactant are present in the separate liquid. In a case where a solution having cutting ability with respect to the bond between the solid phase and the protein portion is selected as the eluent, proteins are mixed into the separate liquid. In a case where a solution which does not have cutting ability with respect to the bond between the solid phase and the protein portion is selected as the eluent, proteins are not substantially contained in the separate liquid.

(Purification)

Depending on the method of analyzing the sugar chain, the labeled sugar chain may be purified by removing unnecessary substances from the separate liquid. The unnecessary substances may be removed by liquid permeation of the separate liquid through the solid phase for purification, capturing the labeled substance of the sugar chain, and re-eluting the captured labeled substance of the sugar chain.

As an example of the solid phase for purification, a solid phase that captures the labeled sugar chain using a non-covalent bond may be exemplified. Specifically, a silica gel column, an amino column, or other normal phase solid phases can be used.

Other examples of the solid phase for purification include a solid phase that captures the labeled sugar chain by a covalent bond may be exemplified. In this manner, the degree of purification of the labeled sugar chain can be improved in a case where proteins are mixed. Specifically, a polymer having a hydrazide group can be used as the solid phase carrier for purification. In the separate liquid, the isolated sugar chain is in an equilibrium state between a cyclic hemiacetal type and an acyclic aldehyde type, the aldehyde group-CHO specifically reacts with the hydrazide group-NH—NH₂ to form a stable bond —C═N—NH—. In this manner, the isolated sugar chain can be captured by the solid phase carrier for purification. In the re-isolation, a mixed solvent of an acid and an organic solvent or a mixed solvent of an acid, water, and an organic solvent is brought into contact with the solid phase carrier to cause a reaction. The pH of the mixed solvent may be in a range of 2 to 9, in a range of 2 to 7, or in a range of 2 to 6. It is preferable that the reaction is carried out in the vicinity of neutrality from weak acidity from the viewpoint of suppressing hydrolysis of the sugar chain such as releasing a sialic acid residue. However, strong acid conditions with a low pH are also accepted.

[Analyzing Step]

The labeled substance of the sugar chain prepared by the method of the present embodiment can be analyzed qualitatively and/or quantitatively using a known method such as a mass analysis method (such as MALDI-TOF MS), chromatography (such as high performance liquid chromatography or HPAE-PAD chromatography), or electrophoresis (such as capillary electrophoresis). In the sugar chain analysis, various databases (for example. GlycoMod, Glycosuite, or SimGlycan (registered trademark)) can be used.

By means of the analysis of the glycoprotein sugar chain as described above, it becomes possible to accelerate research and development of antibody pharmaceutical products; sugar chain modification analysis of antibody pharmaceutical products which is performed during manufacture or quality assurance; analysis of glycoproteins in a specimen such as serum or the like which is performed during the retrieve and research of sugar chain biomarkers; sugar chain analysis of stem cells; analysis of sugar chains in an electrophoretic gel band; and sugar chain analysis of plant tissues.

[Kit]

According to a first embodiment, the present invention provides a kit for preparing a glycoprotein sugar chain, including: a solid phase for fixing a glycoprotein; a container for isolating and labeling a sugar chain by holding the solid phase; and a sugar chain-isolating enzyme.

The kit of the present embodiment is used to perform the method of preparing the glycoprotein sugar chain described above. The kit of the present embodiment may include protocol information for using a kit. The protocol information for using a kit may be in the form of printed matter on which the method of preparing the glycoprotein sugar chain of the present invention is shown or may be access information that enables access to information on the web on which the method described above is shown.

In addition, the kit of the present embodiment may further include any one or all of a pre-treatment agent containing a surfactant, a deglycosylation promoter containing an acid-derived anionic surfactant, a labeling reagent, a solid phase for clean-up, and a container to be filled with a solid phase for clean-up.

Here, the surfactant contained in the pre-treatment agent and the acid-derived anionic surfactant contained in the deglycosylation promoter may be formed of the same compound. In this case, the pre-treatment agent and the deglycosylation promoter are not distinguished from each other and may be accommodated in one container.

As the container used for performing isolation and labeling of the sugar chain by holding the solid phase for fixing the glycoprotein or a container to be filled with the solid phase for clean-up, a column, a multi-well plate, a filter plate, or a micro-tube may be used. Among these, a spin column is preferable. The spin column may further include a collection tube for recovering a separate liquid that is subjected to solid-liquid separation by centrifugation. The container may be contained in the kit in a state of being filled with the solid phase or may be contained as an item separate from the solid phase.

The solid phase for fixing the glycoprotein is a solid phase containing, in the surface thereof, binding functional groups such as a specific binding non-covalent binding group (a hydrogen binding group or an ion binding group) which can be bonded to the glycoprotein; and a covalent binding group. Examples of the solid phase include a cation exchange carrier, a hydrophobic interaction carrier, and an inorganic carrier and do not include a solid phase used for merely holding glycoproteins such as an electrophoretic gel and a membrane for transfer.

The solid phase may be an inorganic carrier. In a case where the carriers are inorganic carriers, for example, some carriers are not isolated by a sugar chain-isolating enzyme. Accordingly, appearance of unnecessary signals is easily suppressed during analysis of an isolated sugar chain.

In a case where the glycoprotein is an antibody, the solid phase may contain, in the surface thereof, a ligand selected from the group consisting of protein A, protein G, protein L, protein H, protein D), and protein Arp. In this manner, it is possible to prepare and analyze a sugar chain sample having excellent throughput properties with respect to an antibody with a particularly high importance for sugar chain analysis.

In addition, the labeling reagent may contain 2-aminobenzamide, a reducing agent, and a solvent. Further, the 2-aminobenzamide, the reducing agent, and the solvent are accommodated in containers different from one another and may be mixed at the time of use.

The sugar chain can be isolated from the glycoprotein by the kit of the present embodiment without performing the treatment of decomposing the protein portion. Accordingly, the time taken for the sugar chain isolation treatment can be greatly shortened. Further, it is possible to make the sugar chain-isolating enzyme act easily during the sugar chain isolation treatment.

Further, in a case where the kit contains the labeling reagent, the sugar chain can be extremely rapidly prepared from the glycoprotein using a sample for analysis (labeled form) by adding a labeling reagent to the container without separating the isolated product.

[Device]

According to the first embodiment, the present invention provides a device for preparing a glycoprotein sugar chain including: a container holding portion which holds a container in which a sample that is fixed to a solid phase and contains a glycoprotein is accommodated; and a reagent introducing unit which introduces a reagent into the container, in which the reagent introducing unit includes a sugar chain-isolating enzyme introducing unit which introduces a sugar chain-isolating enzyme into the container and a labeling reagent introducing unit which introduces a labeling reagent into the container. Further, the configuration of the device described below is merely an example and the scope of the present invention is not restricted by this configuration.

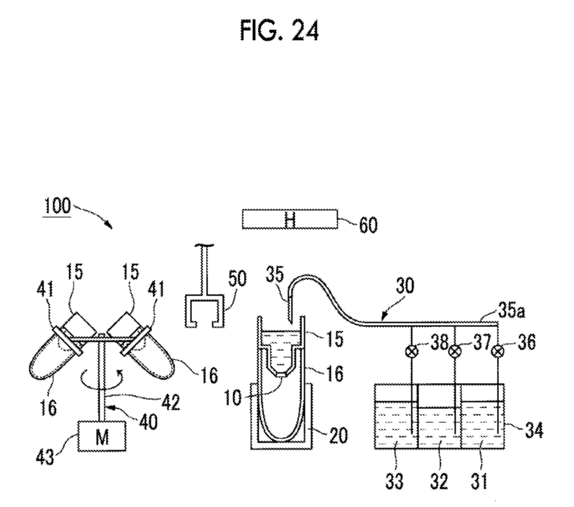

FIG. 24 is a view schematically illustrating the device of the present embodiment. A device 100 includes a container holding portion 20 which holds a container 15 in which a sample that is fixed to a solid phase 10 and contains a glycoprotein is accommodated; and a reagent introducing unit 30 which introduces a reagent into the container 15, in which the reagent introducing unit 30 includes a sugar chain-isolating enzyme introducing unit 35 which introduces a sugar chain-isolating enzyme 31 into the container 15 and a labeling reagent introducing unit 35 which introduces a labeling reagent 32 into the container. In the present example, the sugar chain-isolating enzyme introducing unit and the labeling reagent introducing unit are configured of the same member.

The container holding portion 20 is used for holding the reaction container 15 to accommodate the sample containing the glycoprotein fixed to the solid phase 10. The form of the container holding portion 20 holding the container 15 is not particularly limited, and a form in which the container is held by fitting most of the container into holding holes or holding pores of the container holding portion 20 may be exemplified. Other examples of the form include a form in which an engaging concave portion (engaging convex portion) of the container is engaged with an engaging convex portion (engaging concave portion) of the container holding portion so that the container is held; and a form in which the container is clamped by clamping portions of the container holding portion so that the container is held.

The reagent introducing unit 30 is used to introduce liquids into the container 15 held by the container holding portion 20. The reagent introducing unit 30 includes at least the sugar chain-isolating enzyme introducing unit 35 which introduces the sugar chain-isolating enzyme 31 used in the isolation step; and the labeling reagent introducing unit 35 which introduces the labeling reagent 32 used in the labeling step.

In the example of FIG. 24, the reagent introducing unit 30 includes a tank 34 in which the sugar chain-isolating enzyme 31, the labeling reagent 32, and a pre-treatment agent/deglycosylation promoter 33 are accommodated; a liquid supply pipe 35a which supplies each reagent accommodated in the tank 34; valves (36, 37, and 38) which control the liquid supply of each reagent; and an introducing unit 35 which introduces each reagent into the container 15.

The sugar chain-isolating enzyme 31 and the labeling reagent 32 are added to the same reaction container 15 by the sugar chain-isolating enzyme introducing unit 35 and the labeling reagent introducing unit 35. The form of the reagent introducing unit 30 introducing a liquid into the reaction container 15 is not particularly limited, and a form of supplying a liquid into the reaction container 15 through a tubular member from liquid supply sources (31, 32, and 33) storing the liquid to be supplied may be exemplified. Other examples thereof include a form of injecting a liquid collected by the tubular member into the reaction container.

The sugar chain-isolating enzyme introducing unit 35 and the labeling reagent introducing unit 35 may be configured as constituent members which are separate and independent from each other. In this case, the sugar chain-isolating enzyme 31 and the labeling reagent 32 may be sequentially introduced in this order or may be introduced at the same time. The labeling reagent introducing unit 35 may be automatically controlled, and in a case where both reagents are sequentially introduced, the timing of the operation of the labeling reagent introducing unit 35 may be controlled based on the reaction time or the like required for the isolation step.

Alternatively, the sugar chain-isolating enzyme introducing unit 35 and the labeling reagent introducing unit 35 may be configured as the same constituent member. In this case, the sugar chain-isolating enzyme 31 and the labeling reagent 32 may be introduced in a state of being mixed with each other or may be sequentially introduced in this order. In a case where both reagents are sequentially introduced, the timing of supplying the labeling reagent, that is, the timing of functioning the liquid introducing unit as the labeling reagent introducing unit may be controlled based on the reaction time or the like required for the isolation step.

The device 100 may further include a solid-liquid separating unit 40 which performs solid-liquid separation on the contents in the container 15. In a case where the device 100 includes the solid-liquid separating unit 40, the solid-liquid separating unit 40 separates a solid and a liquid from the contents contained in the container 15. The solid is a substance that remains in the container 15. Substantially, the solid includes the solid phase 10 and a substance fixed to the solid phase 10. In this case, a container (for example, a spin column or a micro-plate provided with a filter) including a filter that is capable of performing solid-liquid separation is used as the container 15. Further, a recovery container 16 (for example, a collection tube or a collection plate) may be mounted on the container 15 and then used. In this case, the container holding portion 20 may be configured by containing a recovery container holding portion that holds the recovery container 16 mounted on the container 15. In the example of FIG. 24, the recovery container holding portion and the container holding portion 20 may be configured of the same member.

The specific separation system of the solid-liquid separating unit 40 is not particularly limited, and any of centrifugal filtration, vacuum filtration, and pressurized filtration may be used. In the example of FIG. 24, the separation system of the solid-liquid separating unit 40 is centrifugal filtration. The solid-liquid separating unit 40 includes a rack 41 which holds a container 15 (or 16); a drive shaft 42; and a motor 43.

As shown in the example of FIG. 24, the solid-liquid separating unit 40 may be configured as a constituent member independent from the container holding portion 20 on which the isolation step and the labeling step are performed. In this case, the device 100 may include a container transfer unit 50 that automatically transfers the container 15 (and 16) to the solid-liquid separating unit 40 front the container holding portion 20. The container transfer unit 50 may be configured such that only the container 15 is transferred during the transfer of the container 15 (and 16) or may be configured such that the container 15 is transferred in a state in which the recovery container 16 is mounted on the container 15. The container transfer unit 50 may be configured by containing an arm which is operated to grasp, release, and move the container 15 directly or indirectly (in other words, through the recovery container 16); and an arm control unit which controls the operation of the arm.

A liquid is recovered by the recovery container 16 by operating the solid-liquid separating unit 40. Accordingly, the separate liquid containing the labeled substance of the sugar chain can be recovered in the recovery container 16 by performing vacuum filtration, pressurized filtration, or centrifugation from the reactant (that is, the contents in the reaction container after the reaction) in the reaction container obtained by introducing the sugar chain-isolating enzyme 31 and the labeling reagent 32. For example, in the step of preparing a sample containing the glycoprotein fixed to the solid phase 10, the glycoprotein fixed to the solid phase 10 can be left in the container 15 and the liquid component can be discarded in the recovery container 16 from a preparation obtained by bringing the sample containing the glycoprotein into contact with the solid phase and capturing the glycoprotein.

Further, in a case where the device 100 includes the solid-liquid separating unit 40, the above-described liquid introducing unit 35 may be configured such that the cleaning solution can be introduced into the container 15. In this manner, the cleaning solution can permeate through the container 15.

The device 100 may further include a temperature adjusting unit 60 which adjusts the temperature of the contents in the container 15. In a case where the device 100 includes the temperature adjusting unit 60, the temperature adjusting unit 60 may have at least a heater function. The temperature adjusting unit 60 heats the container 15 to the temperatures respectively required for the isolation step and the labeling step. Further, the device 100 may be configured such that an open space communicating with the space in the reaction container is ensured. In this manner, since the solvent in the container 15 is evaporated in a case where the isolation step is performed in an open system, the concentration at which the sugar chain isolation efficiently proceeds can be easily provided regardless of the amount of the glycoprotein. Further, since the isolation reaction is carried out along with the solvent removal, the time for the solvent removal step separately from the isolation step becomes unnecessary so that the sugar chain can be rapidly prepared.

The device 100 may include a liquid transfer unit 50 which automatically transfers the separate liquid containing the labeled substance of the sugar chain recovered in the recovery container by the solid-liquid separation after the labeling step to the column for purification in which the solid phase for purification is accommodated. The column for purification may be provided on the above-described solid-liquid separating unit 40.

In the device 100, at least any of or preferably all of the constituent parts (for example, the liquid introducing unit 35, the arm 50, the solid-liquid separating unit 40, the temperature adjusting unit 60, or the liquid transfer unit 50) which can be operated may be automatically controlled. In this manner, it is possible to rapidly prepare the sugar chain of the glycoprotein.

### EXAMPLES

Hereinafter, the present invention will be described in detail with reference to examples. However, the present invention is not limited to the following examples.

### Comparative Example 1

(Sugar Chain Isolation Due to Trypsin Digestion and Sugar Chain Labeling after Purification of Isolated Sugar Chain)

1 μL of a 1 M ammonium bicarbonate aqueous solution and 1 μL of a 120 mM dithiothreitol aqueous solution were added to 10 μL of an antibody solution containing 1 mg/mL of human IgG (manufactured by Sigma-Aldrich Co., LLC.) in water, and then the solution was allowed to stand at 60° C. for 30 minutes. Next, 2 μL of a 120 mM iodoacetamide aqueous solution was added thereto, and then the solution was allowed to stand at room temperature (25° C.) for 60 minutes under light shielding conditions. Subsequently, 4 μL of a 3 mg/mL of trypsin solution (manufactured by Sigma-Aldrich Co., LLC.) was added thereto, and trypsin digestion was performed at 37° C. for 16 hours. Next, the trypsin was inactivated by treating the solution at 100° C. for 5 minutes.

Next, 2.5 μl, of a 0.5 mU/mL PNGase F solution (manufactured by Takara Bio Inc.) was added thereto, and the sugar chain isolation reaction was performed thereon at 50° C. for 10 minutes so that the sugar chain was isolated. In the mixed solution after the reaction, the isolated sugar chain was captured using sugar chain purification kit BlotGlyco (registered trademark) beads (manufactured by Sumitomo Bakelite Co., Ltd.), and then re-isolation (purification of the isolated sugar chain) and labeling with 2-aminobenzamide (hereinafter, also referred to as “2AB”) were performed, thereby obtaining a separate liquid containing a crude 2AB-labeled sugar chain.

Next, acetonitrile was added to the obtained separate liquid containing a crude 2AB-labeled sugar chain, the solution was applied to the clean-up column and cleaned, the excess labeling reagent was removed, and the solution was eluted with pure water, thereby obtaining a separate liquid containing a purified 2AB-labeled sugar chain. The time taken from the trypsin digestion until the purified 2AB-labeled sugar chain was obtained was approximately 30 hours.

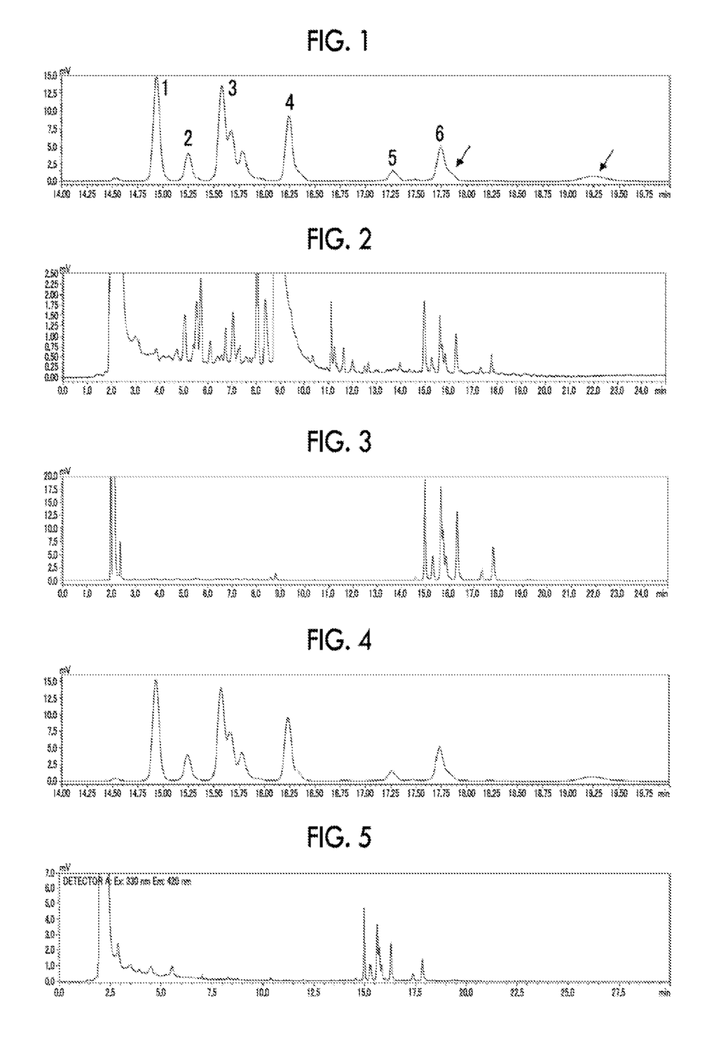

Next, the 2AB-labeled sugar chain was detected by HPLC. The obtained HPLC spectrum is shown in FIG. 1. As shown in FIG. 1, as the peaks of the 2AB-labeled sugar chain, peaks indicated by the numbers of 1 to 6 were detected together with the peaks indicated by arrows in the figure. The peaks indicate by arrows are derived from a sialo-sugar chain. Hereinafter, each peak of the 2AB-sugar chain is noted using the numbers 1 to 6 in FIG. 1. Further, the area ratio of each peak in a case where the sum of peak areas from the peak number 1 to the peak number 6 is set to 100 is listed in Table 1. Between the peaks of the sialo-sugar chain indicated by the arrows in FIG. 1, the area ratio of the peak detected by overlapping the peak indicated by the number 6 due to elution slightly later than the peak indicated by the number 6 is added to the area ratio indicated by the number 6.

TABLE 1

| Peak number | Area ratio (%) |
|---|---|
| 1 | 22.58 |
| 2 | 6.76 |
| 3 | 38.52 |
| 4 | 17.49 |
| 5 | 2.30 |
| 6 | 11.14 |

### Example 1

(Sugar Chain Isolation and Sugar Chain Labeling on Protein A-Sepharose)

A solution obtained by dissolving 20 μg of human IgG (manufactured by Sigma-Aldrich Co., LLC.) in a phosphoric acid buffer solution (PBS) was provided for 25 μL of Protein A-Sepharose (manufactured by GE Healthcare) and cleaned with PBS.

Next, 9 μL of a 0.5 mU/mL PNGase F solution (manufactured by Takara Bio Inc.) and 1 μL of a 1 M ammonium bicarbonate aqueous solution were added thereto, the sugar chain isolation reaction was performed at 50° C. for 15 minutes, and the sugar chain was isolated.

Subsequently, 50 μL of a 2AB solution (solution obtained by mixing 50 mg of 2-aminobenzamide, 60 mg of sodium cyanoborohydride, 300 μL of acetic acid, and 700 μL of dimethyl sulfoxide) was added thereto to cause a reaction at 60° C. for 2 hours.

Next, centrifugation was performed using a desktop centrifuge, thereby obtaining a separate liquid containing a crude 2AB-labeled sugar chain. Acetonitrile was added to the obtained separate liquid containing a crude 2AB-labeled sugar chain, the solution was applied to the monolith silica spin column and cleaned, and the solution was eluted with 50 μL of pure water, thereby obtaining a separate liquid containing a purified 2AB-labeled sugar chain.

Next, HPLC measurement was performed on the obtained 1 μL of separate liquid containing a purified 2AB-labeled sugar chain under the conditions listed in Table 2.

TABLE 2

| | | |
|---|---|---|
| Column | | Waters ACQUITY UPLC BEH Glycan |
| Mobile phase | A Solution | 0.1% formic acid, 40% acetonitrile |
| | | aqueous solution |
| | B solution | 0.1% formic acid, 90% acetonitrile |
| | | aqueous solution |

| | |
|---|---|
| Elution conditions | B: 0% (0 min) → B: 100% (20 min) |
| Flow rate | 0.2 mL/min |
| Column temperature | 40° C. |
| Detection | Fluorescent. detector (excitation wavelength of |
| | 320 nm, fluorescent wavelength of 400 nm) |

The A solution and the B solution of Table 2 were liquids respectively constituting a mobile phase, and the polarity of the mobile phase was adjusted by mixing the A solution and the B solution. Further, in Table 2, the description of “B: a % (T₁ min)→B: b % (T₂ min)” means that the concentration of the B solution was changed from a % to b % during (T₂−T₁) minutes. Here, T₁, T₂, a, and b each represent a real number. Further. “%” in Table 2 indicates % by volume.

The obtained HPLC spectrum is shown in FIG. 2. As shown in FIG. 2, it was confirmed that the 2AB-labeled sugar chain was detected. Further, the time taken for all steps (from adsorption of an antibody to Protein A-Sepharose to HPLC detection) was approximately 3 hours.

### Example 2

(Sugar Chain Isolation and Sugar Chain Labeling on Protein A-Binding Monolith Silica)

The same operation as in Example 1 was performed except that the solid phase was changed into monolith silica (the use volume was approximately 25 μL) bonded to Protein A.

The obtained HPLC spectrum is shown in FIG. 3. As shown in FIG. 3, it was confirmed that the 2AB-labeled sugar chain was detected. Further, the noise in which the retention time was detected to be shorter than that of the 2AB-labeled sugar chain in Example 1 was not almost detected in the present example, and the noise was reduced in the detection range of the 2AB-labeled sugar chain. Further, the time taken for all steps (from adsorption of an antibody to Protein A-monolith silica to HPLC detection) was approximately 3 hours.

### Example 3

(Sugar Chain Isolation and Sugar Chain Labeling Using Deglycosylation Promoter on Protein A-Binding Monolith Silica Subjected to Pre-Treatment)

The same operation as in Example 1 was performed except that the solid phase was changed into monolith silica (the use volume was approximately 5 μL) bonded to Protein A, 500 μL of a 0.4 mass % sodium N-lauroylsarcosine (hereinafter, also referred to as “NLS”) aqueous solution was allowed to permeate before an action of PNGase F, and 9 μL, of the PNGase F solution and 1 μL of the 1 M ammonium bicarbonate aqueous solution used for isolating the sugar chain were changed into 2 μL of the 0.2 M ammonium bicarbonate aqueous solution (the final concentration of NLS after being mixed with the PNGase F solution was 0.2% by mass) containing 2 μL of the PNGase F solution and NLS.

The obtained HPLC spectrum is shown in FIG. 4. As shown in FIG. 4, it was confirmed that the 2AB labeled sugar chain was detected. Further, in the present example, a sialo-sugar chain corresponding to the sialo-sugar chain indicated by the arrows in FIG. 1 was also detected.

Further, the area ratio of each peak in a case where the sum of peak areas from the peak number 1 to the peak number 6 is set to 100 is listed in Table 3. Between the peaks of the sialo-sugar chain corresponding to the sialo-sugar chain indicated by the arrows in FIG. 1, the area ratio of the peak detected by overlapping the peak indicated by the number 6 due to elution slightly later than the peak indicated by the number 6 is added to the area ratio indicated by the number 6.

TABLE 3

| Peak number | Area ratio (%) |
|---|---|
| 1 | 23.02 |
| 2 | 6.52 |
| 3 | 40.24 |
| 4 | 17.08 |
| 5 | 2.32 |
| 6 | 10.23 |

In the present example, despite the fact that the time taken for all steps (from adsorption of an antibody to Protein A-monolith silica to HPLC detection) was only 3 hours, an excellent recovery rate which was not inferior to that in Reference Example 1 described below was achieved. Further, the peaks of the sialo-sugar chain corresponding to the sialo-sugar chain indicated by the arrows in FIG. 1 were detected with preferable intensity similar to Comparative Example 1 in which trypsin digestion was performed and then sugar chain isolation was performed.

### Reference Example 1

(Sugar Chain Isolation Using Deglycosylation Promoter and Sugar Chain Labeling after Sugar Chain Purification on Protein A-Binding Monolith Silica)

The same operation as in Example 1 was performed and the sugar chain was isolated except that the solid phase was changed into monolith silica (the use volume was approximately 5 μL) bonded to Protein A and 9 μL of the PNGase F solution and 1 μl of the 1 M ammonium bicarbonate aqueous solution used for isolating the sugar chain were changed into 2 μL of the 0.2 M ammonium bicarbonate aqueous solution (the final concentration of NLS after being mixed with the PNGase F solution was 0.2% by mass) containing 2 μL of the PNGase F solution and NLS.

Next, centrifugation was performed using a desktop centrifuge, thereby obtaining a separate liquid containing a crude isolated sugar chain. The isolated sugar chain was captured by bringing the separate liquid into contact with sugar chain purification kit BlotGlyco (registered trademark) beads (manufactured by Sumitomo Bakelite Co., Ltd.), and then re-isolation (purification of the isolated sugar chain) of the captured sugar chain and labeling with 2-aminobenzamide (2AB) were performed, thereby obtaining a separate liquid containing a crude 2AB-labeled sugar chain. Acetonitrile was added to the obtained separate liquid containing a crude 2AB-labeled sugar chain, the solution was applied to the monolith silica spin column and cleaned, the excess labeling reagent was removed, and the solution was eluted with 50 μL of pure water, thereby obtaining a separate liquid containing a purified 2AB-labeled sugar chain.

The obtained HPLC spectrum is shown in FIG. 5. As shown in FIG. 5, it was confirmed that the 2AB-labeled sugar chain was detected. Further, in the present reference example, the sialo-sugar chain corresponding to the sialo-sugar chain indicated by the arrows in FIG. 1 was also detected.

Further, the area ratio of each peak in a case where the sum of peak areas from the peak number 1 to the peak number 6 is set to 100 is listed in Table 4. In the introduction of the area ratio of the number 6, between the peaks of the sialo-sugar chain corresponding to the sialo-sugar chain indicated by the arrows in FIG. 1, the area ratio of the peak detected by overlapping the peak indicated by the number 6 due to elution slightly later than the peak indicated by the number 6 is added from the peak portion indicated by the number 6.

TABLE 4

| Peak number | Area ratio (%) |
|---|---|
| 1 | 26.96 |
| 2 | 6.42 |
| 3 | 38.32 |
| 4 | 15.86 |
| 5 | 2.91 |
| 6 | 9.54 |

As listed in Table 4, an excellent recovery rate was achieved. However, in the present reference example, the time taken for all steps (from adsorption of an antibody to Protein A-monolith silica to HPLC detection) was 7 hours.

### Example 4

(Sugar Chain Isolation and Sugar Chain Labeling Using Deglycosylation Promoter on Protein A-Binding Monolith Silica Subjected to Pre-Treatment from Crude Antibody)

The same operation was performed as in Example 3 except that a crude antibody solution obtained by dissolving 20 μg of human IgG (manufactured by Sigma-Aldrich Co., LLC.) in a cell culture solution was used in place of the solution obtained by dissolving 20 μg of human IgG (manufactured by Sigma-Aldrich Co., LLC.) in PBS.

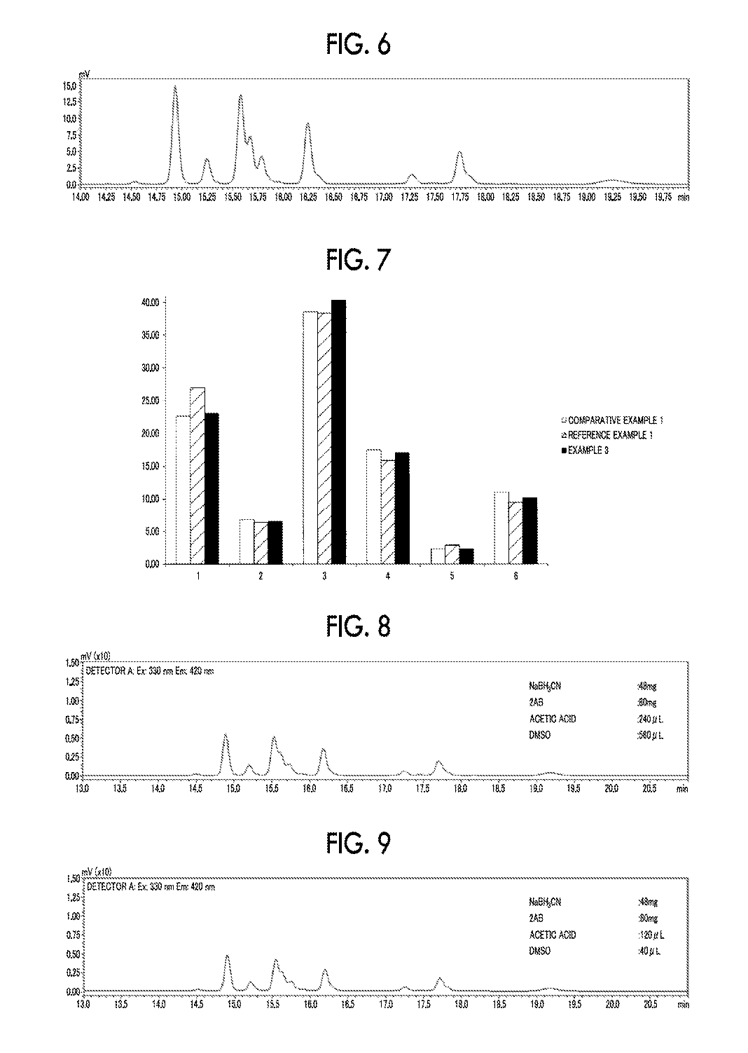

The obtained HPLC spectrum is shown in FIG. 6. As shown in FIG. 6, it was confirmed that the 2AB-labeled sugar chain was detected. Further, in the present example, the sialo-sugar chain corresponding to the sialo-sugar chain indicated by the arrows in FIG. 1 was also detected.

In the present example, despite the fact that an antibody which had not been purified was used, the time taken for all steps (from adsorption of an antibody to Protein A-monolith silica to HPLC detection) was only 3 hours and an excellent spectrum similar to Examples 2 and 3 and Reference Example 1 was detected. Further, the peaks of the sialo-sugar chain corresponding to the sialo-sugar chain indicated by the arrows in FIG. 1 were detected with preferable intensity similar to Comparative Example 1 in which trypsin digestion was performed and then sugar chain isolation was performed.

Further, the area ratio of each peak in a case where the sum of peak areas from the peak number 1 to the peak number 6 is set to 100 is listed in Table 5. Between the peaks of the sialo-sugar chain corresponding to the sialo-sugar chain indicated by the arrows in FIG. 1, the area ratio of the peak detected by overlapping the peak indicated by the number 6 due to elution slightly later than the peak indicated by the number 6 is added to the area ratio indicated by the number 6.

TABLE 5

| Peak number | Area ratio (%) |
|---|---|
| 1 | 22.74 |
| 2 | 6.77 |
| 3 | 39.35 |
| 4 | 16.85 |
| 5 | 2.35 |
| 6 | 11.55 |

[Verification of Reproducibility]

A variation coefficient CV (100×(standard deviation/average value)) of the peak areas and the area ratios of the number 1 to the number 7 was derived by performing the operation of Example 4 three times. The results are listed in Table 6. As listed in Table 6, it was shown that the reproducibility of the preparation method of the example was excellent. In other words, it was shown that the reliability of the preparation method of the example was high.

TABLE 6

| | CV (%) = 100 × (standard |
|---|---|
| Peak number | deviation/average value) |
| 1 | 0.46 |
| 2 | 2.33 |
| 3 | 0.10 |
| 4 | 0.31 |
| 5 | 0.80 |
| 6 | 0.15 |

[Verification of Peak Pattern]

The graph obtained by comparing the peak area ratios from the number 1 to the number 6 of Comparative Example 1 (30 hours for all steps), Reference Example 1 (7 hours for all steps), and Example 3 (three hours) is shown in FIG. 7. In FIG. 7, the horizontal axis indicates the peak number and the vertical axis indicates the peak area ratio. As shown in FIG. 7, it was shown that excellent peak patterns were maintained despite the fact that remarkable time reduction was achieved in Example 3 compared to Comparative Example 1.

### Example 5

The same operation as in Example 1 was performed except that the 2AB solution was changed into a solution obtained by mixing 48 mg of sodium cyanoborohydride, 80 mg of 2-aminobenzamide, 240 μL of acetic acid, and 560 μL of dimethyl sulfoxide. The obtained HPLC spectrum is shown in FIG. 8.

### Example 6

The same operation as in Example 1 was performed except that the 2AB solution was changed into a solution obtained by mixing 48 mg of sodium cyanoborohydride, 80 mg of 2-aminobenzamide, 120 μL of acetic acid, and 40 μL of dimethyl sulfoxide and the reaction time taken for 2AB labeling was set to 40 minutes. The obtained HPLC spectrum is shown in FIG. 9.

### Example 7

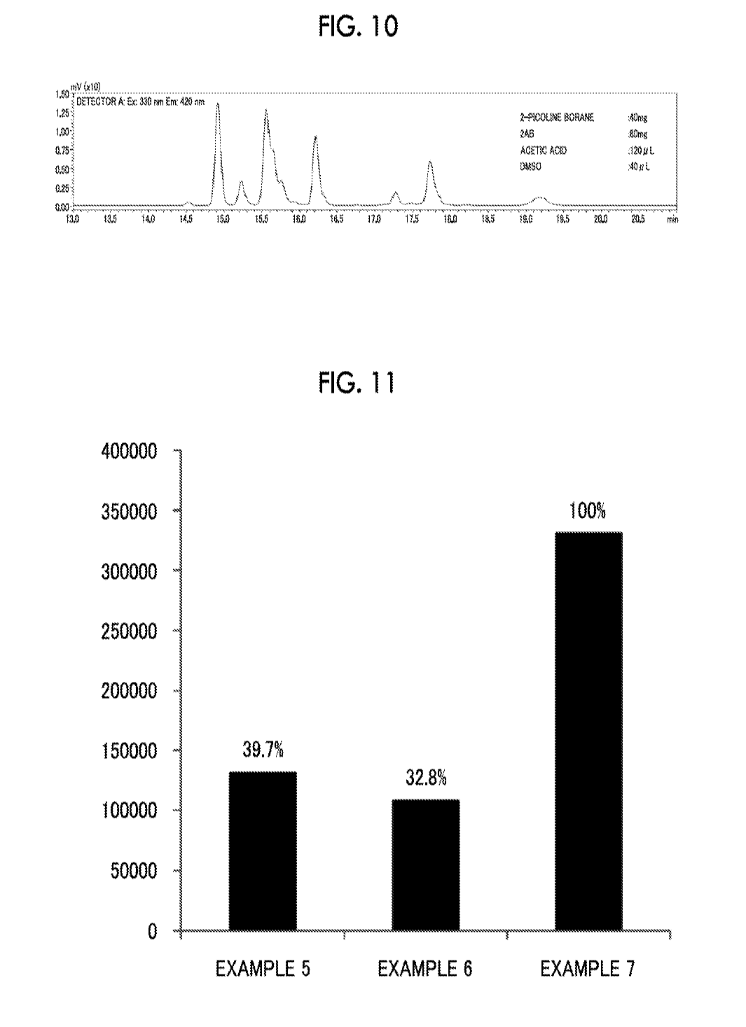

The same operation as in Example 1 was performed except that the 2AB solution was changed into a solution (acetic acid concentration of 75% by volume) obtained by mixing 40 mg of 2-picoline borane, 80 mg of 2-aminobenzamide, 120 μL of acetic acid, and 40 μL of dimethyl sulfoxide and the reaction time taken for 2AB labeling was set to 40 minutes. The obtained HPLC spectrum is shown in FIG. 10.

[Verification of Total Value of Peak Areas (Relationship Between Reducing Agent and Concentration)]

A graph obtained by comparing the total value of the peak areas from the number 1 to the number 6 (see FIG. 1) in the HPLC spectra of Example 5 (2 hours of reaction time, low concentration of NaBH₃CN), Example 6 (40 minutes of reaction time, high concentration of NaBH₃CN), and Example 7 (40 minutes of reaction time, high concentration of picoline borane) is shown in FIG. 11. In FIG. 11, the vertical axis indicates the total value of peak areas. Further, each graph also shows the ratio of the total value of relative peak areas in a case where the total value of peak areas of Example 7 was set to 100%. As shown in FIG. 11, the reaction rate was improved by setting the concentration of NaBH₃CN serving as a reducing agent to be high. Further, it was shown that the effect of improving the reaction rate in a case where picoline borane was used as a reducing agent was extremely high when compared to the case of using NaBH₃CN as a reducing agent.

### Example 8

The same operation as in Example 1 was performed except that the 2AB solution was changed into a solution (acetic acid concentration of 40% by volume) obtained by mixing 40 mg of 2-picoline borane, 80 mg of 2-aminobenzamide, 64 μL of acetic acid, and 96 μL of dimethyl sulfoxide and the reaction time taken for 2AB labeling was set to 40 minutes.

### Example 9

The same operation as in Example 1 was performed except that the 2AB solution was changed into a solution (acetic acid concentration of 50% by volume) obtained by mixing 40 mg of 2-picoline borane, 80 mg of 2-aminobenzamide, 80 μL of acetic acid, and 80 μL of dimethyl sulfoxide and the reaction time taken for 2AB labeling was set to 40 minutes.

### Example 10

The same operation as in Example 1 was performed except that the 2AB solution was changed into a solution (acetic acid concentration of 60% by volume) obtained by mixing 40 mg of 2-picoline borane, 80 mg of 2-aminobenzamide, 96 μL of acetic acid, and 64 μL of dimethyl sulfoxide and the reaction time taken for 2AB labeling was set to 40 minutes.

### Example 11

The same operation as in Example 1 was performed except that the 2AB solution was changed into a solution (acetic acid concentration of 70% by volume) obtained by mixing 40 mg of 2-picoline borane, 80 mg of 2-aminobenzamide, 112 μL of acetic acid, and 48 μL of dimethyl sulfoxide and the reaction time taken for 2AB labeling was set to 40 minutes.

### Example 12

The same operation as in Example 1 was performed except that the 2AB solution was changed into a solution (acetic acid concentration of 80% by volume) obtained by mixing 40 mg of 2-picoline borane, 80 mg of 2-aminobenzamide, 128 μL of acetic acid, and 32 μL of dimethyl sulfoxide and the reaction time taken for 2AB labeling was set to 40 minutes.

### Example 13

The same operation as in Example 1 was performed except that the 2AB solution was changed into a solution (acetic acid concentration of 90% by volume) obtained by mixing 40 mg of 2-picoline borane, 80 mg of 2-aminobenzamide, 144 μL of acetic acid, and 16 μL of dimethyl sulfoxide and the reaction time taken for 2AB labeling was set to 40 minutes.

### Example 14

The same operation as in Example 1 was performed except that the 2AB solution was changed into a solution (acetic acid concentration of 95% by volume) obtained by mixing 40 mg of 2-picoline borane, 80 mg of 2-aminobenzamide, 152 μL of acetic acid, and 8 μL of dimethyl sulfoxide and the reaction time taken for 2AB labeling was set to 40 minutes.

[Verification of Total Value of Peak Areas (Relationship Between Peak Area and Acetic Acid Concentration)]

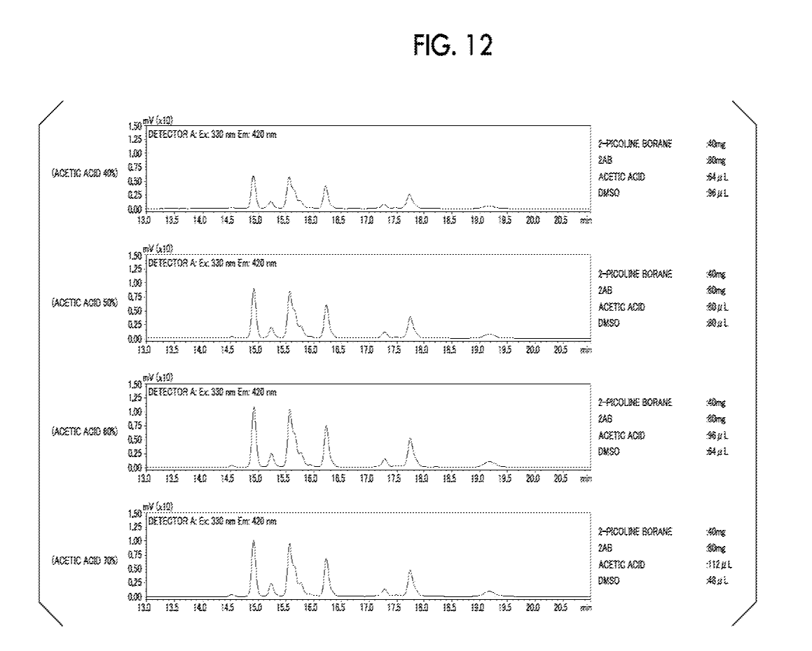

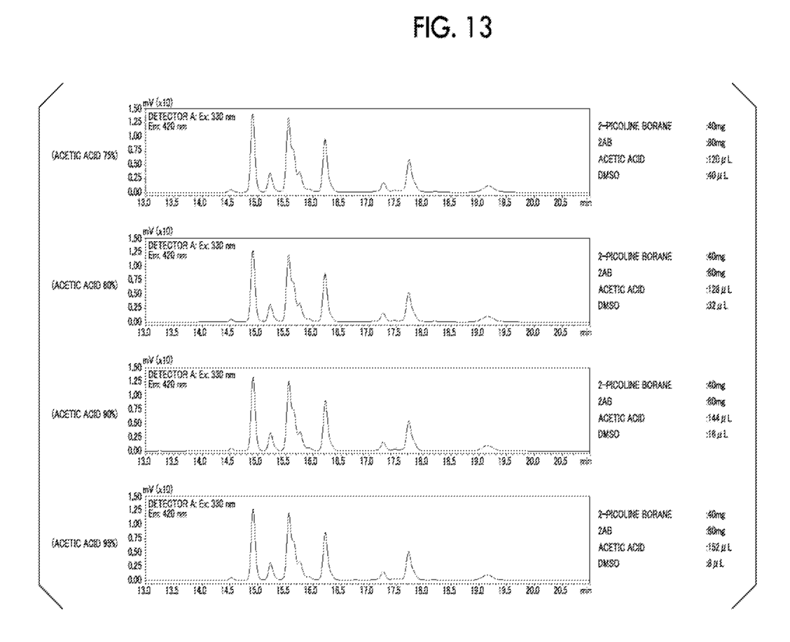

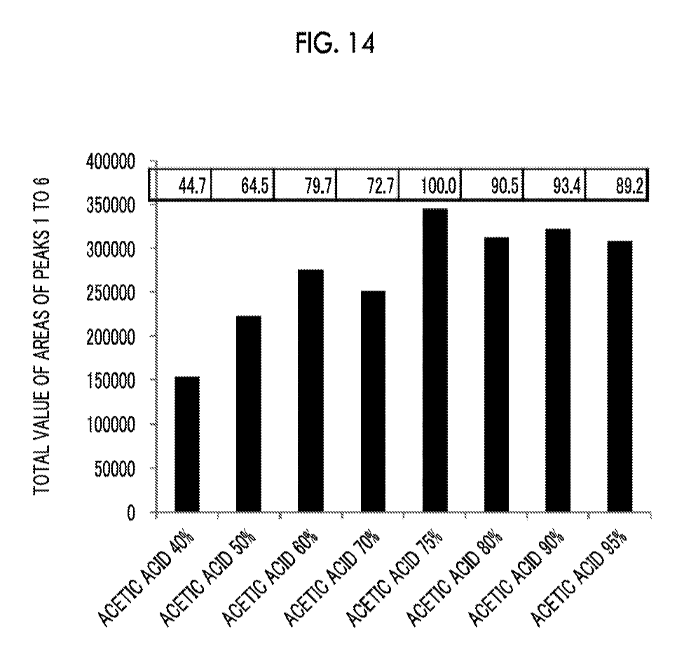

The HPLC spectra obtained in Example 8 (acetic acid concentration of 40% by volume) to Example 14 (acetic acid concentration of 95% by volume) are shown together with the HPLC spectrum of Example 7 (acetic acid concentration of 75% by volume) in FIGS. 12 and 13. A graph obtained by comparing the total value of the peak areas from the number 1 to the number 6 (see FIG. 1) in the HPLC spectra of Examples 7 to 14 is shown in FIG. 14. In FIG. 14, the vertical axis indicates the total value of peak areas and the horizontal axis indicates the acetic acid concentration. Further, each graph also shows the ratio of the total value of relative peak areas in a case where the total value of peak areas of Example 7 was set to 100%.

As illustrated in FIG. 14, even in a case where the acetic acid concentration was 40% by volume, since the ratio of the total value of peak areas was 44.7% of a case where the acetic acid concentration was 75% by volume, it was shown that the effect of improving the reaction rate was high with reference to FIG. 11. It was shown that a further improved reaction rate was stably obtained when the acetic acid concentration was 60% by volume or greater (the value of peak areas which was 70% or greater of a case where the acetic acid concentration was 75% by volume was ensured). Particularly, it was shown that the extremely improved reaction rate was stably obtained when the acetic acid concentration was 75% by volume or greater (the value of peak areas which was approximately 90% or greater of a case where the acetic acid concentration was 75% by volume was ensured).

### Example 15

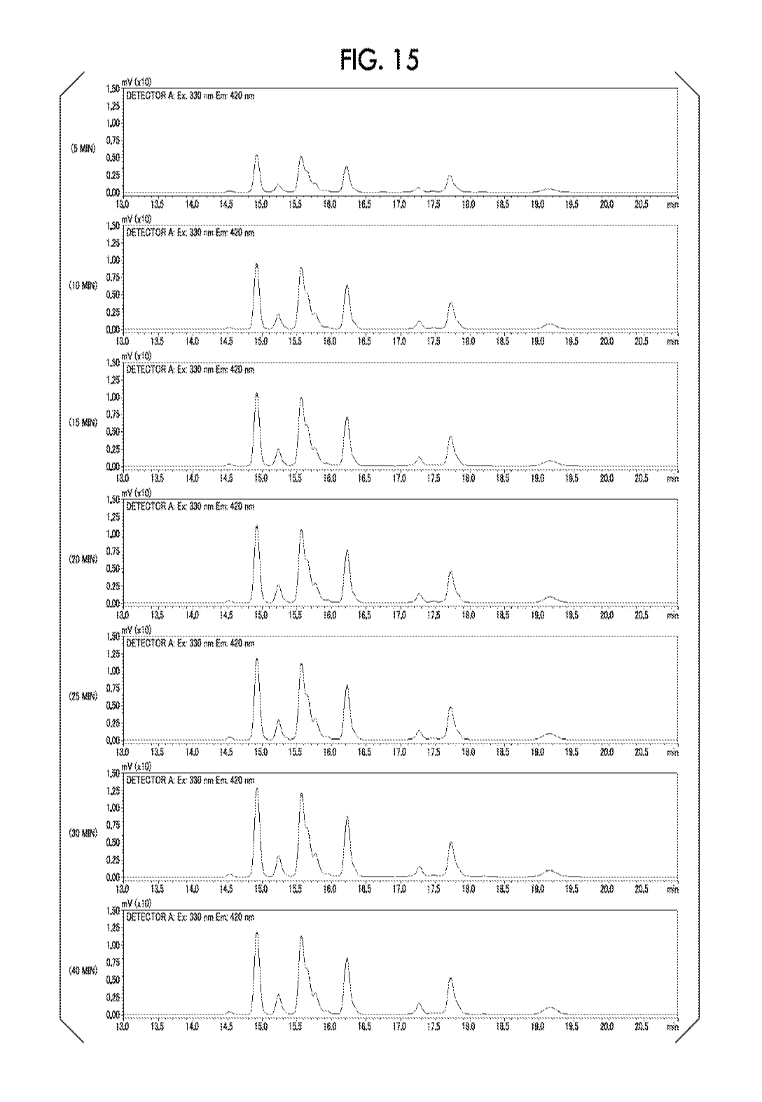

The HPLC spectra were obtained in each case where the reaction time taken for the 2AB labeling was set to 5 minutes, 10 minutes, 15 minutes, 20 minutes, 25 minutes, 30 minutes, and 40 minutes using the same 2AB solution of Example 7 (the solution (acetic acid concentration of 75% by volume) obtained by mixing 40 mg of 2-picoline borane, 80 mg of 2-aminobenzamide, 120 μL of acetic acid, and 40 μL of dimethyl sulfoxide). The obtained HPLC spectra are shown in FIG. 15.

[Verification of Total Value of Peak Areas (Relationship Between Peak Area and Reaction Time)]

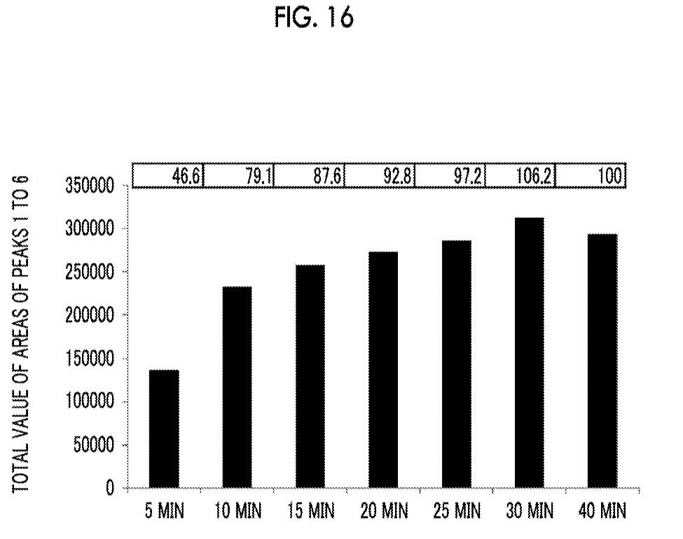

A graph obtained by comparing the total value of the peak areas from the number 1 to the number 6 (see FIG. 1) in the HPLC spectra obtained in Example 15 is shown in FIG. 16. In FIG. 16, the vertical axis indicates the total value of peak areas and the horizontal axis indicates the reaction time. Further, each graph also shows the ratio of the total value of relative peak areas in a case where the total value of peak areas when the reaction time was 40 minutes was set to 100%.

As illustrated in FIG. 16, even in a case where the reaction time was 5 minutes, since the ratio of the total value of peak areas was 46.6% of a case where the reaction time was 40 minutes, it was shown that the effect of improving the reaction rate was high with reference to FIG. 11. It was shown that a further improved reaction rate was stably obtained when the reaction time was 10 minutes or longer (the value of peak areas which was approximately 80% or greater of a case where the reaction time was 40 minutes was ensured). Particularly, it was shown that the extremely improved reaction rate was stably obtained when the reaction time was 25 minutes or longer (the value of peak areas which was approximately 95% or greater of a case where the reaction time was 40 minutes was ensured).

### Comparative Example 2

(Sugar Chain Isolation Due to Trypsin Digestion)

1 μL of a 1 M ammonium bicarbonate aqueous solution and 1 μL of a 120 mM dithiothreitol aqueous solution were added to 10 μL of an antibody solution containing 1 mg/mL of human IgG (manufactured by Sigma-Aldrich Co., LLC.) in water, and then the solution was allowed to stand at 60° C. for 30 minutes. Next, 2 μL of a 120 mM iodoacetamide aqueous solution was added thereto, and then the solution was allowed to stand at room temperature (25° C.) for 60 minutes under light shielding conditions. Subsequently, 4 μL of a 3 mg/mL of trypsin solution (manufactured by Sigma-Aldrich Co., LLC.) was added thereto, and trypsin digestion was performed at 37° C. for 16 hours. Next, the trypsin was inactivated by treating the solution at 100° C. for 5 minutes.

Next, 2.5 μL of a 0.5 mU/mL. PNGase F solution (manufactured by Takara Bio Inc.) was added thereto, and the sugar chain isolation reaction was performed thereon at 50° C. for 10 minutes so that the sugar chain was isolated. In the mixed solution after the reaction, the isolated sugar chain was captured using sugar chain purification kit BlotGlyco (registered trademark) beads (manufactured by Sumitomo Bakelite Co., Ltd.), and then re-isolation and fluorescent labeling with 2-aminobenzamide (2AB) were performed. The time taken from the trypsin digestion until the labeled and isolated sugar chain was obtained was approximately 30 hours.

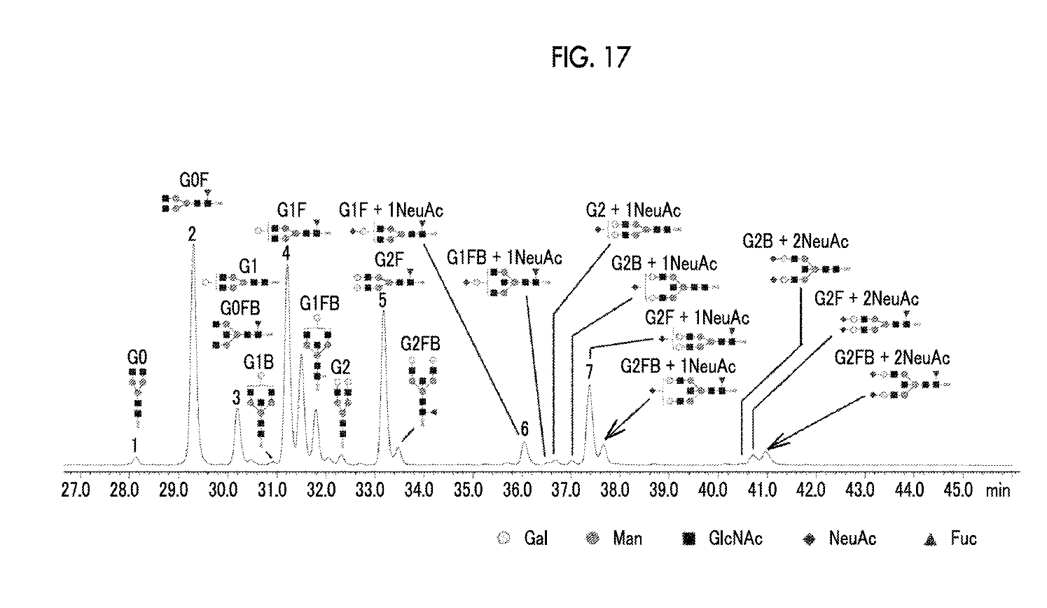

The modified and isolated sugar chain was detected by HPLC. The obtained HPLC spectrum is shown in FIG. 17. FIG. 17 also shows the identification results of each peak. As shown in FIG. 17, peaks of various sialo-sugar chains and neutral sugar chains were detected. Among the sialo-sugar chains, particularly the peaks of sialo-sugar chains indicated by the arrows were detected.

### Comparative Example 3

(Sugar Chain Isolation in Absence of Deglycosylation Promoter without Pre-Treatment)

A solution obtained by dissolving 20 μg of human IgG (manufactured by Sigma-Aldrich Co., LLC.) in PBS was provided for Protein A column (carrier formed by Protein A being fixed to the surface of monolith silica, the volume of carrier: approximately 5 μL. and the volume thereof includes the volume of the silica itself and the volume of mesopores and macropores) and cleaned with PBS. Next, 1.5 μL of a 0.5 mU/mL PNGase F solution (manufactured by Takara Bio Inc.) and 1.5 μL of 0.1 M ammonium bicarbonate solution were added to the Protein A column, the sugar chain isolation reaction was performed at 50° C. for 10 minutes, and the sugar chain was isolated.

Subsequently, 10 μL of a 2AB solution (solution obtained by mixing 50 mg of 2-aminobenzamide, 60 mg of sodium cyanoborohydride, 300 μL of acetic acid, and 700 μL of dimethyl sulfoxide) was added to the Protein A column to cause a reaction at 60° C. for 2 hours. The Protein A column was put into a tube, and centrifugation was performed using a desktop centrifuge, thereby obtaining a 2AB-labeled product. Acetonitrile was added to the obtained 2AB-labeled product, and an eluate was obtained. The eluate was applied to the monolith silica spin column, cleaned, and eluted with 50 μL of pure water, thereby obtaining an eluate.

(HPLC Measurement)

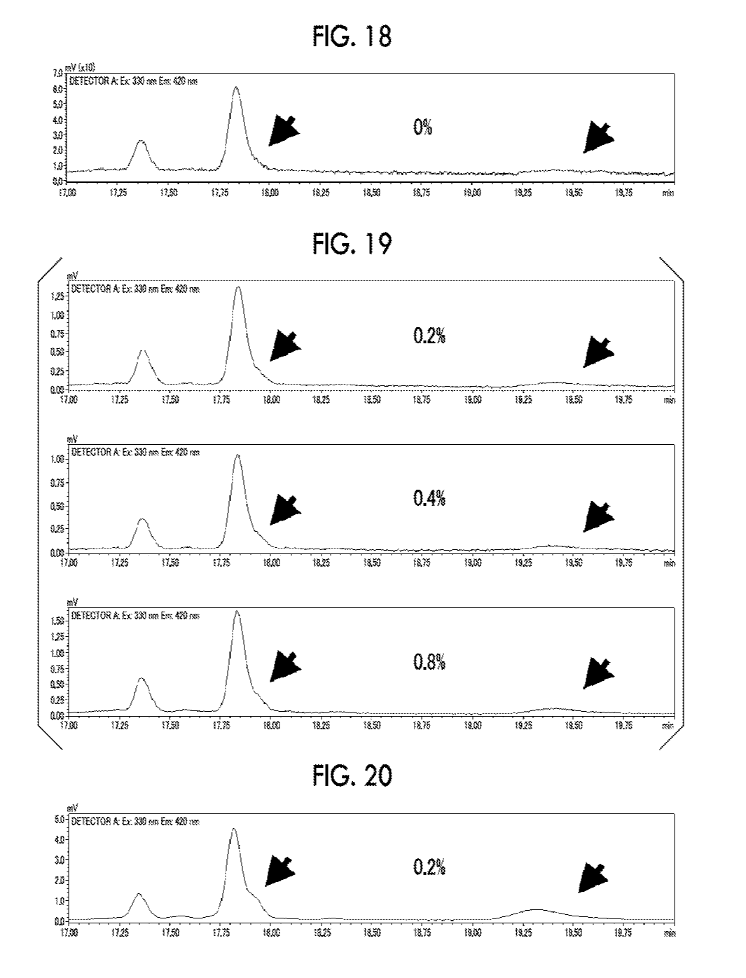

The HPLC measurement was performed on 1 μL of the obtained eluate under the above-described conditions listed in Table 1. The obtained HPLC spectrum is shown in FIG. 18. As shown in FIG. 18, peaks (peaks indicated by arrows in FIG. 8) containing a sialo-sugar chain containing bisecting GlcNAc indicated by arrows in FIG. 17 and a disialo-sugar chain were not detected.

### Reference Example 2

(Sugar Chain Isolation in Presence of Deglycosylation Promoter without Pre-Treatment)

A solution obtained by dissolving 20 μg of human IgG (manufactured by Sigma-Aldrich Co., LLC.) in PBS was provided for Protein A column (carrier formed by Protein A being fixed to the surface of monolith silica, the volume of carrier: approximately 5 μL) and cleaned with PBS.

Next, 1.5 μl, of a 0.5 mU/mL PNGase F solution (manufactured by Takara Bio Inc.) and 1.5 μL of a 0.1 M ammonium bicarbonate solution containing NLS were added to the Protein A column (the final concentration of NLS was 0.2% by mass, 0.4% by mass, or 0.8% by mass), the sugar chain isolation reaction was performed at 50° C. for 10 minutes, and the sugar chain was isolated.

Subsequently, 10 μL of a 2AB solution (solution obtained by mixing 50 mg of 2-aminobenzamide, 60 mg of sodium cyanoborohydride, 300 μL of acetic acid, and 700 μL of dimethyl sulfoxide) was added to the Protein A column to cause a reaction at 60° C. for 2 hours.

Subsequently, the Protein A column was put into a tube, and centrifugation was performed using a desktop centrifuge, thereby obtaining a 2AB-labeled product. Acetonitrile was added to the obtained 2AB-labeled product, and an eluate was obtained. The eluate was applied to the monolith silica spin column, cleaned, and eluted with 50 μL of pure water, thereby obtaining an eluate. The HPLC measurement was performed on the obtained eluate under the same conditions as those in Comparative Example 3.

The obtained HPLC spectra are shown in FIG. 19. As shown in FIG. 19, peaks of sialo-sugar chains indicated by arrows in FIG. 17 were detected despite the fact that peptide digestion was not performed in Reference Example 2.

### Example 16

(Sugar Chain Isolation in Presence of Deglycosylation Promoter with Pre-Treatment)

A solution obtained by dissolving 20 μg of human IgG (manufactured by Sigma-Aldrich Co., LLC.) in PBS was provided for Protein A column (carrier formed by Protein A being fixed to the surface of monolith silica, the volume of carrier: approximately 5 μL) and cleaned with PBS.

Next, 500 μL of a 0.2 mass % NLS aqueous solution was allowed to permeate as the pre-treatment, 1.5 μL of a 0.5 mU/mL PNGase F solution (manufactured by Takara Bio Inc.) and 1.5 μL of a 0.1 M ammonium bicarbonate solution containing NLS were added to the Protein A column (the final concentration of NLS was 0.2% by weight), the sugar chain isolation reaction was performed at 50° C. for 10 minutes, and the sugar chain was isolated.

Subsequently, 10 μL of a 2AB solution (solution obtained by mixing 50 mg of 2-aminobenzamide, 60 mg of sodium cyanoborohydride, 300 μL. of acetic acid, and 700 μL of dimethyl sulfoxide) was added to the Protein A column to cause a reaction at 60° C. for 2 hours.

Subsequently, the Protein A column was put into a tube, and centrifugation was performed using a desktop centrifuge, thereby obtaining a 2AB-labeled product. Acetonitrile was added to the obtained 2AB-labeled product, and an eluate was obtained. The eluate was applied to the monolith silica spin column, allowed to permeate, cleaned, and eluted with 50 μL of pure water, thereby obtaining an eluate. The time taken from the sugar chain isolation reaction until the isolated sugar chain was obtained was approximately 3 hours. The HPLC measurement was performed on the obtained eluate under the same conditions as those in Comparative Example 3.

The obtained HPLC spectrum is shown in FIG. 20. As shown in FIG. 20, peaks of the sialo-sugar chain indicated by arrows in FIG. 17 were detected despite the fact that peptide digestion was not performed in the present example. Further, since the pre-treatment was performed, peaks of the sialo-sugar chain were detected with stronger intensity than that of Reference Example 2.

### Example 17

(Examination of Recovery Rate Depending on Variation in Concentration of Pre-Treatment Agent)

The same operation as in Example 16 was performed except that the concentration of NLS used for the pre-treatment was changed to 0.4% by weight or 0.8% by weight, and the sugar chain was detected by HPLC.

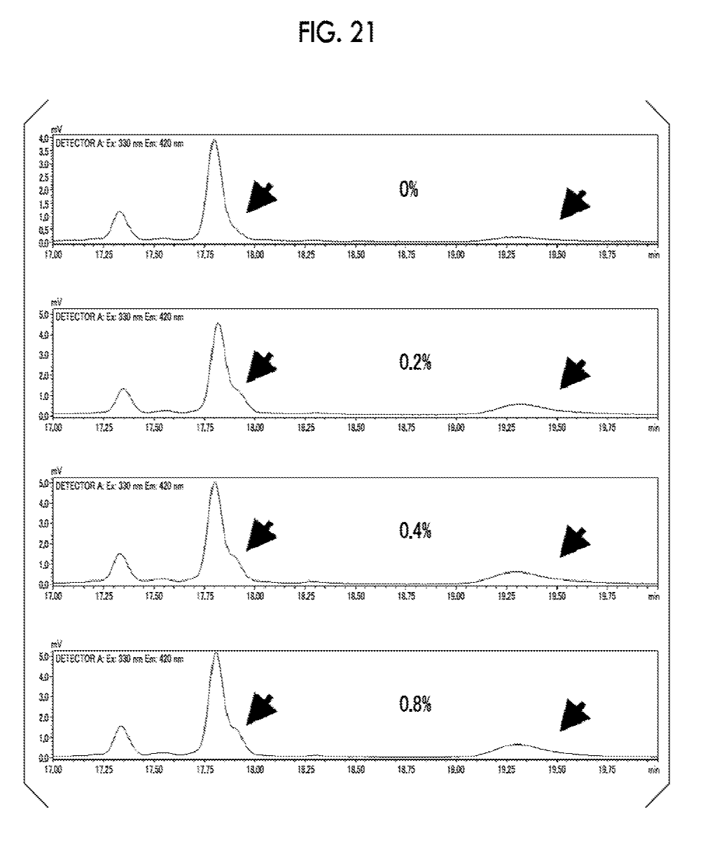

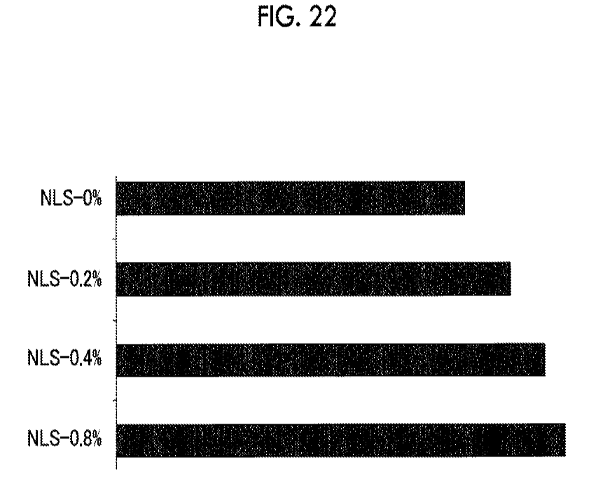

The obtained HPLC spectra are shown in FIG. 21. As shown in FIG. 21, the results of a case where the concentration of NLS in the pre-treatment step was 0% by mass (corresponding to Reference Example 2) and a case where the concentration of NLS was 0.2% by mass (corresponding to Example 16) are shown together. Further, a graph relatively showing the total area of peaks in each HPLC of FIG. 21 is shown in FIG. 22. As shown in FIG. 22, the recovery rate of the sugar chain was improved by performing the pre-treatment.

### Example 18

(Examination of Recovery Rate Depending on Variation in Amount of Pre-Treatment Agent)

The same operation as in Example 16 was performed except that the concentration of NLS of the NLS solution used for the pre-treatment was set to 0.4% by mass and the amount of the NLS solution was set to 10 μL, 50 μL, 200 μL, or 500 μL, and the sugar chain was detected by HPLC.

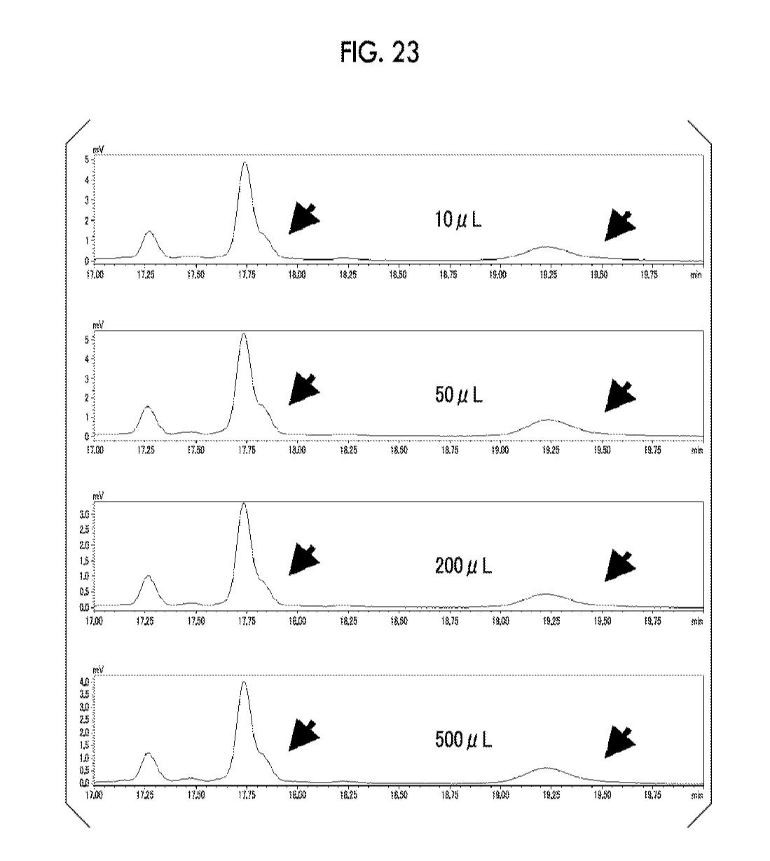

The obtained HPLC spectra are shown in FIG. 23. Further, the ratio (%) of the area of each peak to the total area of peaks indicated by the number 1 to the number 7 (see FIG. 17) in the obtained HPLC spectra is shown in Table 7. The area of the peaks indicated by the number 7 includes the area of the peaks (peaks indicated by arrows which are detected by overlapping the peaks of the number 7) containing the sialo-sugar chain eluted slightly after the peak indicated by the number 7. Therefore, the recovery rate of the sialo-sugar chain is determined based on the peak area of the number 7 in Table 7. As listed in Table 7, it became evident that the recovery rate of the sialo-sugar chain was increased by particularly using the 0.4 mass % NLS solution by an amount of 50 μL or greater.

TABLE 7

| Peak number | 10 μL | 50 μL | 200 μL | 500 μL |
|---|---|---|---|---|
| 1 | 0.59 | 0.55 | 0.55 | 0.57 |
| 2 | 23.05 | 22.74 | 22.97 | 22.92 |
| 3 | 6.68 | 6.77 | 6.40 | 6.68 |
| 4 | 40.48 | 40.31 | 40.10 | 40.40 |
| 5 | 16.98 | 16.88 | 17.25 | 17.08 |
| 6 | 2.33 | 2.22 | 2.40 | 2.24 |
| 7 | 9.89 | 10.52 | 10.32 | 10.11 |

### INDUSTRIAL APPLICABILITY

According to the present invention, it is possible to provide a technique for rapidly preparing a labeled sugar chain from a glycoprotein.

### REFERENCE SIGNS LIST

100: device for preparing glycoprotein sugar chain
10: solid phase
15: container
16: recovery container
20: container holding portion
30: reagent introducing unit
31: sugar chain-isolating enzyme
32: labeling reagent
33: pre-treatment agent/deglycosylation promoter
34: tank
35: sugar chain-isolating enzyme introducing unit (labeling reagent introducing unit)
35a: liquid supply pipe
36, 37, 38: valve
40: solid-liquid separating unit
41: rack
42: drive shaft
43: motor
50: container transfer unit (liquid transfer unit)
60; temperature adjusting unit

1-22. (canceled)

23. A method for preparing a glycoprotein sugar chain, comprising:
an isolation step of acting a sugar chain-isolating enzyme on a sample which contains a glycoprotein fixed to a solid phase in a container to obtain an isolated product which contains a sugar chain; and
a labeling step of adding a labeling reagent to the isolated product in the container to obtain a labeled product which contains a labeled substance of the sugar chain, wherein
the glycoprotein is an antibody, and the solid phase includes a ligand selected from the group consisting of protein A, protein G, protein L, protein H, protein D, and protein Arp, in the surface thereof.

24. The method for preparing a glycoprotein sugar chain according to claim 23,
wherein the isolation step is performed in an open system and under heating conditions.

25. The method of preparing a glycoprotein sugar chain according to claim 23,
wherein the labeling reagent contains 2-aminobenzamide, a reducing agent, and a solvent.

26. The method of preparing a glycoprotein sugar chain according to claim 25,
wherein the reducing agent is picoline borane.

27. The method of preparing a glycoprotein sugar chain according to claim 25,
wherein the solvent contains a protic compound.

28. The method of preparing a glycoprotein sugar chain according to claim 27,
wherein the solvent further contains an aprotic compound having a boiling point higher than that of the protic compound.

29. The method of preparing a glycoprotein sugar chain according to claim 23, further comprising:
a separation step of performing solid-liquid separation after the isolation step to obtain a separate liquid which contains the isolated product.

30. The method of preparing a glycoprotein sugar chain according to claim 23, further comprising:
a separation step of performing solid-liquid separation after the labeling step to obtain a separate liquid which contains the labeled substance of the sugar chain.

31. A method for preparing a glycoprotein sugar chain, comprising:
an isolation step of acting a sugar chain-isolating enzyme on a sample which contains a glycoprotein fixed to a solid phase in a container to obtain an isolated product which contains a sugar chain; and
a labeling step of adding a labeling reagent to the isolated product in the container to obtain a labeled product which contains a labeled substance of the sugar chain, wherein
wherein the isolation step is performed in the presence of a deglycosylation promoter containing an acid-derived anionic surfactant.

32. The method for preparing a glycoprotein sugar chain according to claim 31, further comprising:
a pre-treatment step of bringing a pre-treatment agent containing a surfactant into contact with the sample before the isolation step.

33. The method for preparing a glycoprotein sugar chain according to claim 31,
wherein the acid-derived anionic surfactant is selected from the group consisting of a carboxylic acid type anionic surfactant, a sulfonic acid type anionic surfactant, a sulfuric acid ester type anionic surfactant, and a phosphoric acid ester type anionic surfactant.

34. A kit for preparing a glycoprotein sugar chain, comprising:
a solid phase for fixing a glycoprotein;
a container for isolating and labeling a sugar chain by holding the solid phase; and
a sugar chain-isolating enzyme, wherein
the solid phase includes a ligand selected from the group consisting of protein A, protein G, protein L, protein H, protein D, and protein Arp, in the surface thereof.

35. A kit for preparing a glycoprotein sugar chain, comprising:
a solid phase for fixing a glycoprotein;
a container for isolating and labeling a sugar chain by holding the solid phase;
a deglycosylation promoter which contains an acid-derived anionic surfactant; and
a sugar chain-isolating enzyme.

36. A device for preparing a glycoprotein sugar chain comprising:
a container holding portion which holds a container in which a sample that contains a glycoprotein fixed to a solid phase is accommodated; and
a reagent introducing unit which introduces a reagent into the container, wherein
the glycoprotein is an antibody, and the solid phase includes a ligand selected from the group consisting of protein A, protein G, protein L, protein H, protein D, and protein Alp, in the surface thereof, and
the reagent introducing unit includes a sugar chain-isolating enzyme introducing unit which introduces a sugar chain-isolating enzyme into the container and a labeling reagent introducing unit which introduces a labeling reagent into the container.

37. The device for preparing a glycoprotein sugar chain according to claim 36, further comprising:
a solid-liquid separating unit which performs solid-liquid separation on the contents in the container.

38. A device for preparing a glycoprotein sugar chain comprising:
a container holding portion which holds a container in which a sample that contains a glycoprotein fixed to a solid phase is accommodated; and
a reagent introducing unit which introduces a reagent into the container,
wherein the reagent introducing unit includes a sugar chain-isolating enzyme introducing unit which introduces a sugar chain-isolating enzyme into the container, a labeling reagent introducing unit which introduces a labeling reagent into the container, and a deglycosylation promoter introducing unit which introduces a deglycosylation promoter which contains an acid-derived anionic surfactant into the container.
